(19) United States
(12) Patent Application Publication
(10) Pub. No.: US 2012/0039881 A1
(43) Pub. Date: Feb. 16, 2012

(54) COMPOSITIONS AND METHODS FOR TREATING NEUROLOGICAL DISORDERS

(75) Inventors: Lin Mei, Evans, GA (US); Wen-Cheng Xiong, Evans, GA (US); Ran-Sook Woo, Augusta, GA (US); Xiaoming Li, Augusta, GA (US)

(73) Assignee: Georgia Health Sciences University Research Institute, Inc.

(21) Appl. No.: 13/278,417

(22) Filed: Oct. 21, 2011

Related U.S. Application Data
(62) Division of application No. 12/624,035, filed on Nov. 23, 2009.
Application No. 12/624,035 is a continuation-in-part of application No. PCT/US2008/064742, filed on May 23, 2008.
(60) Provisional application No. 60/931,419, filed on May 23, 2007.

(51) Int. Cl. A61K 39/395; A61K 38/18; A61K 39/00; A61P 25/22; A61P 25/18; A61P 25/08; A61P 25/24; A61K 38/45; A61P 25/00

(52) U.S. Cl. 424/133.1; 424/94.5; 514/9.6; 424/184.1

(57) ABSTRACT

Methods and compositions for modulating GABA release in a subject are provided. A preferred embodiment provides a composition containing an effective amount of an ErbB4 ligand to enhance or promote GABA release, i.e., GABAergic transmission. The ErbB4 ligand can be an agonist ligand or an antagonist ligand depending on the disorder to be treated. Methods for treating neurological disorders are also provided. Representative disorders that can be treated include, but are not limited to schizophrenia, epilepsy, depression and anxiety, insomnia, stroke, pain, bipolar, autism, or a combination thereof. By increasing GABA release a sedative effective can be induced in the subject. Methods for inducing a stimulatory effect in a subject are also provided. In these methods, an effective amount of an ErbB4 antagonist ligand is administered to the subject to reduce or inhibit GABA release in the subject.

### CROSS-REFERENCE TO RELATED APPLICATIONS

This application is a divisional application of U.S. application Ser. No. 12/624,035, filed on Nov. 23, 2009, which is a continuation-in-part application of PCT/US2008/064742, filed with the U.S. Receiving Office of the Patent Cooperation Treaty on May 23, 2008, which claims benefit of and priority to U.S. Provisional Patent Application No. 60/931,419 filed on May 23, 2007, and where permissible all of these are incorporated by reference in their entirety.

### FIELD OF THE INVENTION

The invention is generally directed to methods and compositions for treating one or more symptoms of a neurological disorder, in particular schizophrenia, epilepsy, depression and anxiety, insomnia, stroke, pain, bipolar, autism and combinations thereof.

### BACKGROUND OF THE INVENTION

Both NRG1 and ErbB4 are susceptibility genes of schizophrenia, a mental disorder that affects 1% of the total population. Schizophrenic patients express abnormal levels of NRG1 and ErbB4 in regions increasingly implicated in schizophrenia.

Epilepsy is the most prevalent chronic neurologic condition. In developed countries, its incidence is 30-50 per 100 000 population per year and the prevalence is approximately 5-8 cases per 1 000 population. The rapid growth of health care expenditures has led to increased interest in economic evaluation of health care programs.

Gamma-aminobutyric acid (GABA) and glutamic acid are major neurotransmitters which are involved in the regulation of brain neuronal activity.

GABA is a major inhibitory neurotransmitter in the mammalian central nervous system. Meythaler et al., Arch. Phys. Med. Rehabil.; 80:13-9 (1999). Imbalances in the levels of GABA in the central nervous system can lead to conditions such as schizophrenia, spastic disorders, convulsions, and epileptic seizures. As described in U.S. Pat. No. 5,710,304, when GABA levels rise in the brain during convulsions, seizures terminate. GABA agonists are believed to be beneficial to schizophrenic patients.

Because of the inhibitory activity of GABA and its effect on convulsive states and other motor dysfunctions, the administration of GABA to subjects to increase the GABA activity in the brain has been tried. Because it is difficult to develop and administer a GABA compound which is able to cross the blood brain barrier utilizing systemic administration of GABA compounds, different approaches have been undertaken including making GABA lipophilic by conversion to hydrophobic GABA amides or GABA esters, and by administering activators of L-glutamic acid decarboxylase (GAD) whose levels vary in parallel with increases or decreases of brain GABA concentration, and which have been reported to increase GABA levels. Additional therapies for modulating GABA concentrations in vivo are needed.

Thus, it is an object of the invention to provide methods and compositions for treating one or more symptoms of a neurological disorder.

It is another object to provide methods and compositions to enhance or promote GABAergic transmission in a subject in need thereof.

It is still another object of the invention to provide methods and compositions for inhibiting or reducing GABAergic transmission in a subject.

### SUMMARY OF THE INVENTION

Methods and compositions for modulating GABA release in a subject are provided. A preferred embodiment provides a composition containing an effective amount of an ErbB4 ligand to enhance or promote GABA release, i.e., GABAergic transmission. The ErbB4 ligand can be an agonist ligand or an antagonist ligand depending on the disorder to be treated. Representative disorders that can be treated include, but are not limited to schizophrenia, epilepsy, depression and anxiety, insomnia, stroke, pain, bipolar, autism, or a combination thereof.

Exemplary agonist ligands include NRG1, variants thereof, antibodies to ErbB4, antibody fragments that bind to ErbB4, and small molecules that mimic NRG1. Exemplary antagonist ligands include the extracellular domain of ErbB4 and fusion proteins thereof, antibodies or antibody fragments that bind to NRG1, and small molecules that inhibit the interaction between NRG1 and ErbB4. The extracellular domain of ErbB4 binds to endogenous NRG1 and thereby reduces or inhibits GABA release.

Methods for treating neurological disorders are also provided. Preferred methods include administering an effective amount of an ErbB4 agonist ligand to a subject in need thereof to promote or enhance GABA release in the subject. By increasing GABA release a sedative effect can be induced in the subject.

Methods for inducing a stimulatory effect in a subject are also provided. In these methods, an effective amount of an ErbB4 antagonist ligand is administered to subject to reduce or inhibit GABA release in the subject.

### BRIEF DESCRIPTION OF THE DRAWINGS

FIG. 1A is a bar graph of percent ErbB4 positives for puncta rings and neuropils in coronal sections of prefrontal cortex. +/−SEM, n=60 for puncta rings and n=10 for neuropils of 20 independent sections. FIGS. 1B and 1C are bar graphs of percent cluster colocalization of coronal sections of prefrontal cortex stained with anti-ErbB4 antibody and anti-GAD65 (G1166) (FIG. 1B) and anti-VGAT (131003) antibodies (FIG. 1C).

FIG. 2A is a line graph of [³H]GABA release fraction/total fraction versus time (mins). Cortical slices were preloaded with [³H]GABA for 30 min in the presence of b-alanine (1 mM), an inhibitor of [³H]GABA uptake by glial cells, aminooxyacetic acid (0.1 mM), an inhibitor of GABA degradation, and nipecotic acid (1 mM), an inhibitor of the GABA transporter in neurons. Basal and depolarization (20 mM KCl)-evoked release of [³H]GABA were monitored sequentially. Controls (open circles) and NRG1 (closed circles). FIG. 2B is a line graph of percent [³H]GABA release versus NRG1 concentration (nM). Basal (open circles) K+ evoked (closed circles). FIG. 2C shows representative traces of mIPSCs in pyramidal neurons in prefrontal cortical slices. FIG. 2D is a line graph of cumulative counts versus mIPSC amplitude (pA). Controls (open circles) and NRG1 (closed circles). FIG. 2E is a line graph of cumulative counts versus mIPSC interevent interval (ms). Controls (open circles) and NRG1 (closed circles). FIG. 2F is a bar graph of mIPSC (percent) in control and NRG1 treated pyramidal neurons in prefrontal cortical slices (n=12). Amplitude (clear) and Frequencies (hatched). FIG. 2G is a bar graph of percent eIPSC amplitude in prefrontal cortical slices treated with NRG1 versus control or washout. n=12, *p<0.01. Representative eIPSCs of control, NRG1-treated, or NRG1-treated/washed slices are shown on top. FIG. 2H is a line graph of percent eIPSC amplitude versus NRG1 (nM). n=6, *p<0.05, **p<0.01. FIG. 2I is a bar graph of K⁺ evoked [³H]GABA release (percent) (left axis, clear rectangles) and eIPSC amplitude (percent) (right axis, hatched rectangles) in prefrontal cortical slices treated with NRG1, denature NRG1, or BDNF. n=8 for [³H]GABA release; for eIPSCs, n=6 for control, NRG1, and denatured NRG1, and n=4 for BDNF. *p<0.05, #p<0.05; **p<0.05, ##p<0.01.

FIG. 3A is a line graph of [³H]GABA release (percent) versus NRG1 (nM). Basal (open circles) and K⁺-evoked (closed circles). [³H]GABA-loaded cortical synaptosomes were treated with 5 nM NRG1 with (evoked) or without (basal) 20 mM KCl. [³H]GABA release was assayed 10 min after NRG1 stimulation. Shown are means±SEM of six individual experiments in triplicate. *p<0.05, **p<0.01. FIG. 3B is a line graph (right) with a series of recordings induced by paired stimulus (10 s apart) separated by indicated interpulse intervals (shown at the left). The line graph is IPSC2/IPSC1 versus interspike intervals (ms) of GABAergin transmission in prefrontal cortex treated with NRG1 (solid circles) or controls (open circles). Inset shows the amplitudes of the first and second IPSCs. n=6, *p<0.05.

FIG. 4A is a bar graph showing quantitative analysis of phospho-ErbB4 (p-ErbB4) in GAD65-positive cortical neurons treated with ecto-ErbB4 for 10 min prior to the addition of NRG1 (5 nM, final concentration) for another 10 min. n=7, *p<0.05. FIG. 4B is a line graph of eIPSC amplitude (nA) versus time (min) for cortical slices treated with sequential addition of NRG1 (5 nM) and ecto-ErbB4 (1 g/ml and 2 μg/ml) (all final concentrations). On the top are averaged traces before (a) and after (b) NRG1, and after different dosages of ecto-ErbB4 ([c] and [d], 1 and 2 μg/ml, respectively). FIG. 4C is a bar graph of K⁺-evoked [³H]GABA release (percent, left axis) or eIPSC amplitude (percent, right axis) in control cortical slices with or without NRG1 (1 μg/ml); slices treated with 1 μg/ml ecto-ErbB4 with or without NRG1 (1 μg/ml); and 2 μg/ml ecto-ErbB4 with NRG1 (1 μg/ml) for 10 min prior to assays of [³H]GABA and eIPSCs. n=5 for [³H]GABA release, n=6 for eIPSCs. *p<0.01 and #p<0.01 for [³H]GABA release and eIPSCs, respectively. K⁺-evoked [³H]GABA release (clear rectangles), eIPSC amplitude (hatched rectangles).

FIG. 5A is a bar graph of phospho-ErbB4 (p-ErbB4) in cortical neurons treated with 5 mM AG1478, an inhibitor of ErbB4, or AG879, an inhibitor of ErbB2, for 10 min prior to the addition of NRG1 (5 nM, final concentration). Neurons were fixed and stained with phospho-ErbB4 and GAD65 antibodies, and visualized with Alexa 594 and FITC-coupled secondary antibodies respectively, and quantified. FIG. 5B is a bar graph of K⁺-evoked [³H]GABA release (percent, left axis) or eIPSC amplitude (percent, right axis) in control cortical slices with or without NRG1, and slices treated with 5 mM AG1478 or AG879 with or without NRG1 for 10 min prior to assay of [³H]GABA or eIPSC recording. n=5 for [³H]GABA release, n=6 for eIPSCs. *p<0.05, #p<0.05; **p<0.01, #p<0.01. K⁺-evoked [³H]GABA release (clear rectangles), eIPSC amplitude (hatched rectangles).

FIG. 6A is a line graph of K⁺-evoked [³H]GABA release (percent) versus NRG1 (nM) in ErbB4^{-/-}ht+ cortical slices (▴) and ErbB4^{+/+}ht⁺ (∘). FIG. 6B is bar graph of eIPSC amplitude (percent) in cortical slices from (ErbB4^{+/+} ht⁺) and ErbB4^{-/-}ht⁺ mice with (clear rectangle) or without (hatched rectangle) NRG1. Shown are normalized eIPSC amplitudes. n=6, *p<0.05. The eIPSC amplitudes in ErbB4^{+/+}ht⁺ and ErbB4^{-/-}ht⁺ were 1014±170 and 598±160 pA, respectively. n=17, p<0.01.

FIG. 7A shows a representative trace of spontaneous spikes recorded in loose patch-clamping of PFC pyramidal neurons treated with vehicle (control), 5 nM NRG1, 1 μg/ml ecto-ErbB4 in the absence (top) or presence (bottom) of 20 μM bicuculline. FIG. 7B is a bar graph of spontaneous firing rates (normalized firing rates (%)) for PFC pyramidal neurons treated with vehicle (control), 5 nM NRG1, or 1 μg/ml ecto-ErbB4 in the absence or presence of 20 μM bicuculline. Shown are means±SEM; n=7, *P<0.05 in comparison with control; #P<0.05, in comparison with NRG1. There was no significant difference in firing rates of three groups: bicuculline alone, bicuculline/NRG1, and bicuculline/ecto-ErbB4 (P>0.05).

FIG. 8A is a representative trace of spontaneous firings of pyramidal neurons treated with vehicle (control), NRG1, or NRG1+ecto-ErbB4. FIG. 8B is a bar graph of spontaneous firing (rate/min) in pyramidal neurons treated with vehicle (control), 1 nM NRG1, or 1 nM NRG1+1 μg/ml ecto-ErbB4.

FIG. 9A shows a representative trace of action potentials of PFC layers II-V pyramidal neurons that were generated by a 200-pA suprathreshold somatic current injection in a whole-cell patch-clamping configuration. FIG. 9B is a chart showing the spike amplitude (mV), RMP (mV), input resistance (MΩ), AHP amplitude (mV), and spike width at half amplitude (ms) of pyramidal cells and interneurons.

FIG. 100A shows representative action potentials of a single pyramidal neuron in a whole-cell patch-clamping before (baseline) and after bath application of vehicle (control), or 5 nM NRG1, or 5 nM denatured NRG1, or 1 μg/ml ecto-ErbB4, or 20 μM bicuculline and 5 nM NRG1, or 20 μM bicuculline and 1 μg/ml ecto-ErbB4. FIG. 10B is a bar graph showing change of firing spikes (%) in pyramidal neurons after bath application of vehicle (control), 5 nM NRG1, or 5 nM denatured NRG1, or 1 μg/ml ecto-ErbB4, or vehicle and 20 μM bicuculline, or 20 μM bicuculline and 5 nM NRG1, or 20 μM bicuculline and 1 μg/ml ecto-ErbB4. n=9, *P<0.05, compared with control; #P<0.05, compared with NRG1. FIG. 10C is a line graph showing evoked spikes (normalized firing spikes (%)) of pyramidal neurons versus dose of NRG1 (n=11).

FIG. 11A shows representative evoked firings of pyramidal neurons treated with vehicle (control), NRG1, or NRG1+ecto-ErbB4. FIG. 11B is a bar graph showing evoked spikes (firing spikes) in pyramidal neurons treated with vehicle (control), 1 nM NRG1, or 1 nM NRG1+1 μg/ml ecto-ErbB4.

FIG. 12A is a bar graph of evoked inhibitory postsynaptic current (eIPSC) amplitudes (normalized amplitude (%)) in PV-Cre;ErbB4+/+ and PV-Cre;ErbB4−/− mice (n=8, *P<0.01, compared with control). Control eIPSC amplitudes, shown with white bars, were 2150±128 and 1650±153 pA for PV-Cre;ErbB4+/+ and PV-Cre;ErbB4−/− mice, respectively (n=8, P<0.05). eIPSC amplitudes after NRG1 treatment are shown with hatched bars. FIG. 12B is a line graph showing normalized eISPC amplitude (%) versus dose of NRG1 (nM) for both PV-Cre;ErbB4+/+ (hatched circles) and PV-Cre;ErbB4−/− (∘) PFC (n=5˜8; *P<0.05, compared with mutant PFC).

FIG. 13A is a representative trace of spontaneous firings of control, 5 nM NRG1, or 1 μg/ml ecto-ErbB4 treated pyramidal neurons of PV-Cre;ErbB4+/+ and PV-Cre;ErbB4−/− mice. FIG. 13B is a bar graph showing spontaneous firings (firing rate/min) of control (white bars), 5 nM NRG1 (hatched bars), or 1 μg/ml ecto-ErbB4 (stippled bars) treated pyramidal neurons from PV-Cre;ErbB4+/+ and PV-Cre;ErbB4−/− mice. (n>6 for both PV-Cre;ErbB4+/+ and PV-Cre;ErbB4−/− mice, *P<0.01, compared with control; #P<0.05, compared with PVCre; ErbB4+/+ samples). FIG. 13C shows representative action potentials produced by a 200-pA current before (top) and after (bottom) bath application of 5 nM NRG1 in PFC slices from PV-Cre;ErbB4+/+ and PV-Cre;ErbB4−/− mice. FIG. 13D is a bar graph showing evoked spike frequency (firing spikes) of pyramidal neurons before (control) and after bath application of 5 nM NRG1 in PFC slices from PV-Cre;ErbB4+/+ and PV-Cre;ErbB4−/− mice (n=9; *P<0.05, compared with control; #P<0.05, compared with PV-Cre;ErbB4+/+ samples). Control is shown with white bars and NRG1 treatment is shown with hatched bars.

FIG. 14A is a bar graph of distance traveled (cm) by PV-Cre;ErbB4+/+ (white bar, n=7) and PV-Cre;ErbB4−/− (hatched bar, n=6) mice during a 30 minute open field test (*P<0.05). FIG. 14B is a line graph showing ambulatory counts (total horizontal photobeam breaks) versus time for PV-Cre;ErbB4+/+ (∘) and PV-Cre;ErbB4−/− (hatched circles) mice (repeated measures for genotype, P=0.045 for ambulatory activity). FIG. 14C is a line graph showing stereotypic counts (repetitive breaks of a given beam with intervals of <1 sec) versus time for PV-Cre;ErbB4+/+ (∘) and PV-Cre;ErbB4−/− (hatched circles) mice (repeated measures for genotype, P=0.043). Activity was summated at 5 min intervals over a 30 min period. FIG. 14D is a line graph showing vertical counts (rearing) (the total number of vertical beam breaks) versus time for PV-Cre;ErbB4+/+ (∘) and PV-Cre;ErbB4−/− (hatched circles) mice (repeated measures for genotype, P=0.202). Activity was evaluated as the total number of vertical beam breaks at 5 min intervals over a 30 min period.

FIG. 15A is a line graph showing the number of errors (revisits and omission) of food-restricted PV-Cre;ErbB4+/+ (hatched circles) (n=9) and PV-Cre;ErbB4−/− (∘) (n=10) mice versus number of trials to retrieve all pellets. Total number of errors were significantly higher in PV-Cre;ErbB4−/− mice in both 4-arm and 8-arm tests (for genotype, P=0.002 and P=0.021, respectively). Significant trial effect was observed in 4-arm test (P<0.001), but not in 8-arm test (P=0.290). FIG. 15B is a line graph showing the amount of time (sec) spent by food-restricted PV-Cre;ErbB4+/+ (hatched circles) and PV-Cre;ErbB4−/− (∘) mice versus number of trials to retrieve all pellets (P=0.096 for 4-arm and P=0.085 for 8-arm test). Significant trial effect was observed for both 4-arm and 8-arm tests (P<0.001). FIG. 15C is a line graph showing the number of correct entries (first 4 out of 8 tries (%)) by food-restricted PV-Cre;ErbB4+/+ (hatched circles) and PV-Cre;ErbB4−/− (∘) mice versus the number of trials to retrieve all pellets. Percentage of correct entries within first 4 and 8 entries was significantly lower in mutant mice in 4-arm test (P=0.044) and 8-arm test (P=0.040), respectively. Significant trial effect was observed in both tests (P<0.001 and P=0.002, respectively).

FIG. 16A is a bar graph showing the baseline startle response (startle amplitude) for PV-Cre;ErbB4+/+ (white bar, n=7) and PV-Cre;ErbB4−/− (hatched bar, n=6) mice under no stimulus, 70 dB (−) and startle stimulus, 120 dB (+), respectively; P>0.05). FIG. 16B is bar graph showing pre-pulse inhibition (%) for PV-Cre;ErbB4+/+ (white bar, n=7) and PV-Cre;ErbB4−/− (hatched bar, n=6) mice at three different levels of pre-pulse (75 dB, 80 dB, 85 dB), P=0.004. PPI=100−100%×(PPx/P120), in which PPx was the amplitude of the startle response after each pre-pulse and P120 was basal startle amplitude. FIG. 16C is a bar graph showing pre-pulse inhibition (%) for PV-Cre;ErbB4+/+ and PV-Cre;ErbB4−/− mice at three different levels of pre-pulse (75 dB, white bars) (80 dB, hatched bars), (85 dB, stippled bars), treated with vehicle or 3 mg/kg diazepam. Repeated measures, *P<0.05, #P=0.573. For vehicle treatment, n=10 and 12 for PV-Cre;ErbB4−/− mice and PV-Cre;ErbB4+/+ littermates, respectively; for diazepam treatment, n=9 and 11 for PV-Cre; ErbB4−/− and PV-Cre;ErbB4+/+, respectively.

### DETAILED DESCRIPTION OF THE INVENTION

### I. Definitions

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered. Non-conservative substitutions typically alter the function of the protein.

The terms “individual”, “host”, “subject”, and “patient” are used interchangeably herein, and refer to a mammal, including, but not limited to, murines, simians, humans, mammalian farm animals, mammalian sport animals, and mammalian pets.

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

The term “soluble ErbB4” or “ecto-ErbB4” are used interchangeably and refer to the extracellular domain or ErbB4 or a fusion protein thereof.

### II. Compositions for Treating Neurological and Psychiatric Disorders

It has been discovered that ErbB4, a receptor for NRG1, is present in GABAergic terminals of the prefrontal cortex (PFC), and that NRG1 facilitates evoked release of GABA from slices of the prefrontal cortex, but has no effect on basal GABA release. The potentiation effect of NRG1 requires ErbB4 because it was blocked by the ErbB4 inhibitor AG 1478 and was abolished in cortical slices of ErbB4 mutant mice. In addition, evoked GABA release and eIPSCs in the absence of exogenous NRG1 were blocked by inhibitors of NRG1 signaling, suggesting a role of endogenous NRG1 in regulating GABA neurotransmission.

NRG1 inhibits the activity of pyramidal neurons in the PFC. Both spontaneous firing rates and the frequency of evoked action potentials in pyramidal neurons were reduced by NRG1, but increased by the neutralizing peptide ecto-ErbB4. These effects were blocked by bicuculline, an antagonist of GABAA receptor, indicating GABA-dependence. Ablation of ErbB4 in parvalbumin (PV)-positive neurons blocked NRG1 potentiation of GABAergic transmission and prevented NRG1 from inhibiting pyramidal neuron firing.

It as been discovered that PV-Cre;ErbB4−/− mice showed schizophrenic-like phenotypes including hyperactivity, impaired working memory and PPI deficit that could be ameliorated by diazepam.

Together, these results identify a novel function of NRG1-regulation of GABAergic transmission via presynaptic ErbB4 receptors. The results also indicate that NRG1 plays a critical role in balancing brain activity and identify PV-positive neurons as a major cellular target of NRG1/ErbB4 signaling in regulating synaptic plasticity.

Therefore, one embodiment provides compositions and methods for treating one or more symptoms of a neurological disorder by modulating GABAergic transmission via NRG1 to induce a sedative or stimulatory outcome. A preferred embodiment provides compositions and methods for treating one or more symptoms of schizophrenia, epilepsy, depression and anxiety, insomnia, stroke, pain, bipolar, autism by administering an effective amount of a ErbB4 ligand, for example NRG1 or a variant thereof. Ligand agonists of ErB4 such as NRG1 induce a sedative effect in a subject by potentiating GABAergic transmission.

Another embodiment provides compositions and methods for inducing a stimulatory effect in a subject. Exemplary compositions include ErbB4 ligand antagonists such as ecto-Erb4r or soluble ErbB4. Ligand antagonists inhibit or reduce ErbB4 activity and thus reduce GABAergic transmission. Reduction in GABAergic transmission induces a stimulatory effect.

A. NRG1 and Neurotransmission at Excitatory and Inhibitory Synapses

NRG1 (NRG1), a family of polypeptides that plays an important role in neural development, is implicated in nerve cell differentiation, neuron migration, neurite outgrowth, and synapse formation (Buonanno and Fischbach, Curr Opin Neurobiol, 11:287-296 (2001); Corfas, et al., Nat Neurosci, 7:575-580 (2004); Mei and Xiong, Nat Rev Neurosci, 9:437-452 (2008)). It acts by stimulating the ErbB family of receptor tyrosine kinases ErbB2, 3, and 4. NRG1 binds only to ErbB3 or ErbB4, but not ErbB2. On the other hand, ErbB2 and ErbB4 are most active in response to NRG1 stimulation whereas the kinase activity of ErbB3 is impaired. Thus, ErbB2 and ErbB3 functions by forming heterodimers with each other or with ErbB4, but an ErbB4 homodimer is functional by itself (2). NRG1 and its receptor ErbB tyrosine kinases are expressed not only in the developing nervous system, but also in adult brain.

In the adult, ErbB receptors are concentrated at the postsynaptic density (PSD), presumably via interaction with PDZ domain containing proteins including PSD-95 and erbin (Garcia, et al., Proc Natl Acad Sci USA, 97:3596-3601 (2000); Huang, et al., Neuron, 26:433-455 (2000); Huang, et al., J Biol Chem, 276:19318-19326 (2001); Ma, et al., J Neurosci, 23:3164-3175 (2003)). NRG1 suppresses induction of LTP at Schaffer collateral-CA1 synapses in the hippocampus without affecting basal synaptic transmission (Huang, et al., Neuron, 26:433-455 (2000); Ma, et al., J Neurosci, 23:3164-3175 (2003)). Subsequently, NRG1 was shown to reverse LTP and reduce whole-cell NMDA receptor currents in pyramidal neurons of prefrontal cortex, and was also shown to decrease NMDA receptor-mediated EPSCs in prefrontal cortex slices (Gu, et al., J Neurosci, 25:4974-4984 (2005); Kwon, et al., J Neurosci, 25:9378-9383 (2005)). Interestingly, the NRG1 gene is strongly associated with schizophrenia in diverse populations in Iceland, Scotland, China, Japan, and Korea (Fukui, et al., Neurosci Lett, 396:117-120 (2006); Kim, et al., Am J Med Genet B Neuropsychiatr Genet, 141:281-286 (2006); Stefansson, et al., Am J Hum Genet, 71:877-892 (2002); Stefansson, et al., Am J Hum Genet, 72:83-87 (2003); Yang, et al., Mol Psychiatry, 8:706-709 (2003)).

ErbB4 mRNA is enriched in regions where interneurons are clustered in adult brains (Lai and Lemke, Neuron, 6:691-704 (1991)). GAD-positive neurons from the embryonic hippocampus express ErbB4 (Huang, et al., Neuron, 26:443-455 (2000)). During development, loss of NRG1/ErbB4 signaling alters tangential migration of cortical interneurons, leading to a reduction in the number of GABAergic interneurons in the cortex (Anton, et al., Nat Neurosci, 7:1319-1328 (2004); Flames, et al., Neuron, 44:251-261 (2004)). In adult mice, deletion of ErbB4 in the central nervous system (CNS) resulted in lower levels of spontaneous motor activity, reduced grip strength, and altered cue use in performing a maze task (Golub, et al., Behav Brain Res, 153:159-170 (2004)). The ErbB4 gene is also associated with schizophrenia (Law, et al., Hum Mol Genet, (2006); Nicodemus, et al., Mol Psychiatry, 11:1062-1065 (2006)).

γ-Aminobutyric acid (GABA) is the principal inhibitory neurotransmitter in the mammalian forebrain. GABAergic inhibitory interneurons are essential to the proper functioning of the CNS (McBain and Fisahn, Nat Rev Neurosci, 2:11-23 (2001)). GABAergic dysfunction is implicated in several neurological disorders, including Huntington's chorea, Parkinson's disease, and epilepsy, and in psychiatric disorders such as anxiety, depression, and schizophrenia (Coyle, Biochem Pharmacol, 68:1507-1514 (2004)).

NRG1 has been implicated in many aspects of neural development including neuron migration, axon projection, myelination, synapse formation or up-regulation of neurotransmitter receptor expression (Mei and Xiong, Nat Rev Neurosci, 9:437-452 (2008)). For example, NRG1 has been shown to regulate differentiation of neural cells, neuronal navigation, and neuron survival in developing CNS (Buonanno and Fischbach, Curr Opin Neurobiol, 11:287-296 (2001); Corfas, et al., Nat Neurosci, 7:575-580 (2004)). In the peripheral nervous system, NRG1 signaling is implicated in Schwann cell differentiation and myelination, muscle spindle development, and synapse-specific expression of AChR subunit genes (Adlkofer and Lai, Glia, 29:104-111 (2000); Fischbach and Rosen, Annu Rev Neurosci, 20:429-458 (1997); Hippenmeyer, et al., Neuron, 36:1035-1049 (2002); Si et al., J Biol Chem, 271:19752-19759 (1996)). Interestingly, NRG1 and its receptor ErbB kinases are continuously expressed in various brain regions, including the prefrontal cortex, hippocampus, cerebellum, oculomotor nucleus, superior colliculus, red nucleus, substantia nigra, and pars compacta (Lai and Lemke, Neuron, 6:691-704 (1991); Law, et al., Neuroscience, 127:125-136 (2004); Yau et al., Cereb Cortex, 13:252-264 (2003)). Moreover, ErbB4 colocalizes with PSD-95 and NMDA receptors in hippocampal neurons (Garcia, et al., Proc Natl Acad Sci USA, 97:3596-3601 (2000); Huang, et al., Neuron, 26:443-455 (2000)). Furthermore, NRG1 signaling may be increased by the interaction of ErbB4 with PSD-95 (Huang, et al., Neuron, 26:443-455 (2000)). These observations suggest that NRG1 may play a role in synaptic plasticity, maintenance or regulation of synaptic structure, or some combination thereof in adult brain. It has been found that NRG1 blocks induction of long-term potentiation (LTP) at Schaffer collateral-CA1 synapses (Huang, et al., Neuron, 26:443-455 (2000)). NRG1 can depotentiate LTP at hippocampal CA1 synapses and reduce whole cell NMDA receptor, but not AMPA receptor, currents in prefrontal cortex pyramidal neurons (Gu, et al., J Neurosci, 25:4974-4984 (2005); Kwon, et al., J Neurosci, 25:9378-9383 (2005)). Recently, ErbB4 has been shown to play a key role in activity-dependent maturation and plasticity of excitatory synaptic structure and function (Li, et al., Neuron, (in press) (2007)).

B. NRG1, ErbB4, and Neurological and Psychiatric Disorders

Schizophrenia exhibits familial characteristics, which suggests a strong genetic component. Disturbances in GABAergic neurotransmission have been thought to be a pathologic mechanism of schizophrenia. Postmortem studies of patient brains reveal decreased levels of the mRNA encoding GAD67 (Hashimoto, et al., J Neurosci, 23:6315-6326 (2003)) and the GABA transporter GAT-1 (Ohnuma, et al., Neuroscience, 93:441-448 (1999)). On the other hand, GABA-A receptor mRNA was shown to be increased in the prefrontal cortex (Ohnuma, et al., Neuroscience, 93:441-448 (1999)). Recently, studies of NRG1 have gained much attention because that both NRG1 and its receptor ErbB4 are susceptibility genes of schizophrenia (Mei and Xiong, Nat Rev Neurosci, 9:437-452 (2008); Stefansson, et al., Am J Hum Genet, 71:877-892 (2002); Yang, et al., Mol Psychiatry, 8:706-709 (2003); Norton, et al., Am J Med Genet B Neuropsychiart Genet, 141:96-101 (2006); Silberberg, et al., Am J Med Genet B Neuropsychiatr Genet, 141:142-148 (2006); Law, et al., Hum Mol Genet, 16:129-141 (2007)). Expression of NRG1 and ErbB4 appeared to be altered in the brains of schizophrenic patients (Mei and Xiong, Nat Rev Neurosci, 9:437-452 (2008)). Moreover, null mutation of the NRG1 gene that disrupts expression of various isoforms and the ErbB4 gene causes a spectrum of abnormal behaviors in mice including hyperactivity, disrupted pre-pulse inhibition (PPI) and spatial learning and memory deficits (Barros, et al., Proc Natl Acad Sci USA, 106:4507-4512 (2009); Stefansson, et al., Am J Hum Genet, 71:877-892 (2002); Gerlai, et al., Behav Brain Res, 109:219-227 (2000); Golub, et al., Behav Brain Res, 153:159-170 (2004); Thuret, et al., J Neurochem, 91:1302-1311 (2004); Rimer, et al., Neuroreport, 16:271-275 (2005); O'Tuathaigh, et al., Neuroreport, 17:79-83 (2006); O'Tuathaigh, et al., Neuroscience, 147:18-27 (2007); O'Tuathaigh, et al., Neurosci Biobehav Rev, 31:60-78 (2007)) which are thought to be associated with schizophrenia (Gainetdinov, et al., Trends Neurosci, 24:527-533 (2001); Geyer and Ellenbroek, Prog Neuropsychopharmacol Biol Psychiatry, 27:1071-1079 (2003); Arguello and Gogos, Neuron, 52:179-196 (2006)). Furthermore, treatment of schizophrenia with antiepileptic drugs that target GABAergic transmission has shown positive results (Hosak and Libiger, Eur Psychiatry, 17:371-378 (2002)). However, the data provided herein is the first to disclose or suggest the treatment of neurological disorders such as epilepsy, depression and anxiety, insomnia, stroke, pain, bipolar, autism by modulating the NRG1/ErbB4 signal transduction pathway.

C. Ligands of ErbB4

Compositions for treating one or more symptoms of a neurological disorder containing an ErbB4 ligand are provided. The ErbB4 ligand can be an agonist ligand or an antagonist ligand. An ErbB4 agonist ligand induces or promotes ErbB4 activity and thereby induces or promotes GABAergic transmission which increases local concentrations of GABA. Because GABA is an inhibitory neurotransmitter, increased concentrations of GABA induce or promote a sedative effect in a subject. Representative ErbB4 agonist ligands include but are not limited to NRG1, small molecules that activate ErbB4, and variants thereof.

ErbB4 antagonist ligands include but are not limited to ecto-ErbB4 or soluble ErbB4, or small molecules, or variants thereof. The antagonist ligand induces or promotes a stimulatory response by reducing the amount of GABAergic transmission for example by binding endogenous NRG1.

In certain embodiment, the ErbB4 ligand is a small molecule, for example a molecule of about 500 Daltons. The small molecules can be obtained by screening a library of compounds for binding to ErbB4. Such screening techniques are routine a known in the art.

1. Variants of ErbB4 Ligands

Exemplary variants of ErbB4 ligands include, but are not limited to NRG1 or ecto-ErbB4 polypeptides that are mutated to contain a deletion, substitution, insertion, or rearrangement of one or more amino acids. In one embodiment the variant ErbB4 ligand has the same activity, substantially the same activity, or different activity as a reference NRG1 or ecto-ErbB4 polypeptide, for example a non-mutated NRG1 or ecto-ErbB4 polypeptide.

A variant NRG1 or ecto-ErbB4 polypeptide can have any combination of amino acid substitutions, deletions or insertions. In one embodiment, isolated NRG1 or ecto-ErbB4 variant polypeptides have an integer number of amino acid alterations such that their amino acid sequence shares at least 60, 70, 80, 85, 90, 95, 97, 98, 99, 99.5 or 100% identity with an amino acid sequence of a wild type NRG1 or ecto-ErbB4 polypeptide. In a preferred embodiment, NRG1 or ecto-ErbB4 variant polypeptides have an amino acid sequence sharing at least 60, 70, 80, 85, 90, 95, 97, 98, 99, 99.5 or 100% identity with the amino acid sequence of a wild type murine or wild type human NRG1 or ecto-ErbB4 polypeptide (GenBank Accession Number NRG1: L12261; ErBB4: L07868].

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in NRG1 polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

2. Fusion Proteins

Fusion proteins that contain an ErbB4 binding domain operably linked to a second polypeptide, in particular a heterologous polypeptide. The fusion protein optionally includes peptide or polypeptide linker domain that separates the ErbB4 binding domain from the second polypeptide.

Optionally, the second polypeptide contains a domain that functions to dimerize or multimerize two or more fusion proteins. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. Typically, the second polypeptide contains an Fc domain.

### III. Formulations

Pharmaceutical compositions including ligands of ErbB4 are provided. The pharmaceutical compositions may be for administration by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in unit dosage forms appropriate for each route of administration. The preferred route is oral.

The one or more active agents can be administered as the free acid or base or as a pharmaceutically acceptable acid addition or base addition salt.

Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

1. Formulations for Enteral Administration

In a preferred embodiment the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The ErbB4 ligands may be chemically modified so that oral delivery of the derivative is efficacious. Generally, the chemical modification contemplated is the attachment of at least one moiety to the component molecule itself, where the moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem, 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The ErbB4 ligands can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e. the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

2. Topical or Mucosal Delivery Formulations

Compositions can be applied topically. The compositions can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent™ nebulizer (Mallinckrodt Inc., St. Louis, Mo.); the Acorn II™ nebulizer (Marquest Medical Products, Englewood, Colo.); the Ventolin™ metered dose inhaler (Glaxo Inc., Research Triangle Park, N.C.); and the Spinhaler™ powder inhaler (Fisons Corp., Bedford, Mass.).

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of disclosed compounds, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6:275-283 (1987); and Mathiowitz, et al., J. Appl. Polymer Sci. 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### IV. Methods of Treatment

Methods for treating one or more symptoms of a neurological disorder are provided. Exemplary neurological disorders that can be treated with the disclosed compositions include, but are not limited to schizophrenia, epilepsy, depression and anxiety, insomnia, stroke, pain, bipolar, autism, or a combination thereof. Symptoms that can be treated with the disclosed compounds include, but are not limited to seizures, prepulse inhibition (PPI), hyperactivity, working memory, hallucinations, delusions, disorganized and unusual thinking and speech, impairment in social cognition, paranoia, avolitiori (apathy or lack of motivation), purposeless agitation, and/or other signs of catatonia.

One embodiment provides administering to subject in need thereof an effective amount of an ErbB4 ligand to reduce or inhibit a neurological disorder. In the preferred embodiment, the neurological disorder is reduced or inhibited by reducing or inhibiting symptoms of the disorder.

One embodiment provides administering to subject in need thereof an effective amount of an ErbB4 ligand to increase or decrease GABAergic transmission in the subject. The ErbB4 ligand can be an agonist ligand or an antagonist ligand depending on the disorder to be treated.

Exemplary ErbB4 ligands include, but are not limited to antibodies to ErbB4. The antibodies can be polyclonal, monoclonal, chimeric, humanized, single-chain, or fragments of these antibodies that bind to ErbB4.

The term “ErbB4 ligand” includes agonist and antagonist ligands. Agonist ligands include, but are not limited to NRG1, variants thereof, and fragments of NRG1 or variants thereof that bind ErbB4 and induce or inhibit GABAergic transmission relative a control. A control can be GABAergic transmission in the absence of the ErbB4 ligand. Antagonist ligands include the extracellular domain of ErbB4 (also referred to as soluble ErbB4 and fusion proteins thereof. Methods for producing fusion proteins are known in the art.

The disclosed compositions can be administered to a subject in need thereof alone or in combination with one or more additional therapeutic agents. The additional therapeutic agents are selected based on the condition, disorder or disease to be treated. A description of the various classes of suitable pharmacological agents and drugs may be found in Goodman and Gilman, The Pharmacological Basis of Therapeutics, (11th Ed., McGraw-Hill Publishing Co.) (2005). For example, pharmaceutical compositions containing ligands of ErbB4 can be administered in combination with one or more known therapeutic agents for treating neurological disorders. Therapeutic agents for treating neurological disorders include, but are not limited to, diazepam, methamphetamine, amphetamine and dextroamphetamine, gabapentin, potassium chloride, methylphenidate, clonazepam, modafinil, lamotrigine, aripiprazole, triamcinolone, valproate semisodium, divalproex sodium, phenyloin sodium, lithium, natalizumab, promethazine, reperidone, temazepam, topiramate, prednisone, triamcinolone, and verapamil.

Ligands of ErbB4 can be administered in combination with one or more neurotransmitters such dopamine, acetylcholine and glutamate, and/or therapeutic agents that increase, decrease, or otherwise effect the production or transmission of neurotransmitters.

One embodiment provides a method for increasing GABAergic transmission in a subject by administering to the subject an effective amount of an ErbB4 agonist ligand, for example NRG1, a variant thereof, or an ErbB4 binding fragment thereof. The agonist ligand binds to ErbB4 and promotes or enhances GABA release i.e, GABAerginc transmission. The increase in the inhibitory transmitter GABA induces a sedative effect in the host.

Another embodiment includes administering to a subject in need thereof an effective amount of an ErbB4 antagonist ligand, for example soluble ErbB4 or a fragment thereof that binds to ErbB4 or a fusion protein thereof. The antagonist ligand binds to ErbB4 and inhibits or reduces GABA release, i.e., GABAergic transmission.

Another embodiment includes a method for administering to subject in need thereof, an effective amount of ErbB4 ligand in combination with a second therapeutic agents for treating a neurological disorder.

One embodiment provides a method for treating schizophrenia by administering to subject in need thereof, an effective amount of ErbB4 ligand agonist in combination with a second therapeutic agent such as diazepam.

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Localization of ErbB4 in GABAergic Presynaptic Terminals

Materials and Methods

Reagents and Animals

The NRG1 used is a recombinant polypeptide containing the entire EGF domain of the b-type NRG1 (rHRG b177-244) (Holmes et al., Science, 256:1205-1210 (1992)). It was prepared in 1% bovine serum albumin (BSA). BDNF was a gift from Regeneron Pharmaceuticals. The ectodomain of ErbB4 (aa 1-659, ecto-ErbB4) was subcloned into pC4DNA/Fc to generate pErbB4ex/Fc. Stable HEK293 cells expressing ecto-ErbB4 were generated and cultured in IgG-low medium for condition media collection. ErbB4ex/Fc was purified by a HiTrap column (Amersham). AG1478 and AG879 were from Calbiochem; poly-L-lysine, nipecotic acid, b-alanine and TMPH (2,2,6,6,-Tetramethylpiperidin-4-yl heptanoate) from Sigma; DL-AP5, CNQX, TTX, bicuculline, LY341495, ipratropium, nicergoline, sotalol, metergoline, MDL 72222, RS 23597-190, and L-741742 from Tocris Bioscience; and aminooxyacetic acid from Chemika. When necessary, chemicals were dissolved in dimethylsulfoxide (DMSO, Sigma); the final concentration of DMSO was 0.001% or less when applied to brain slices. Antibodies were from Sigma (GAD65, G1166); Cell Signaling Technology [ErbB4, #4795; p-ErbB4 (Y1284), #4757]; Transduction Labs (phosphotyrosine, 610024); NeoMarkers (ErbB2, MS-303-PO; ErbB3, MS-229-PO); Santa Cruz Biotechnology (ErbB4, sc-283); and Synaptic Systems (VGAT, 131003). ErbB4^{-/-}ht⁺ mice were kindly provided by Martin Gassmann (Tidcombe et al., Proc Natl Acad Sci USA, 100:8281-8286 (2003)). GAD-GFP mice were from the Jackson Lab.

In Situ Hybridization

In situ hybridization was performed essentially as previously described (Simmons et al., J Histotechnol, 12:169-181 (1989)), with minor modifications. Adult Sprague-Dawley rats were perfused for 20 min with 4% paraformaldehyde in 0.1 M sodium borate buffer (pH 9.5). Sagittal sections (30 mm) were cut on a sliding microtome and mounted on gelatin and poly-L-lysine-coated slides. Tissue sections were fixed for 30 min in 10% buffered formalin and washed in 50 mM KPBS prior to prehybridization. ErbB4 sequence #1009-1931 (accession #NM-021687), NRG1 type I/II sequence #345-845 (accession #NM-031588), and NRG1 type III sequence #555-1321 (accession #AF194438) were subcloned in pCRScript. Plasmids were digested with NotI, SpeI, and EcoRI, respectively, for the production of individual antisense RNAs using T7 RNA polymerase. Transcriptions were performed using 125 μCi ³³P-UTP (2000-4000 Ci/mmole, NEN). After hybridization, the sections were defatted in xylene, rinsed in 100% ethanol and then 95% ethanol, air dried, and dipped in NTB2 emulsion (Kodak) diluted 1:1 with water. The slides were exposed for 2-5 weeks and developed in Kodak D-19 developer. All images were captured with a Hamamatsu Orca ER CCD camera using dark-field microscopy on an Olympus BX-51 microscope at 1.25 3 magnification.

Immunostaining

Immunostaining of rat cortical neurons (E17, DIV14) was performed as previously described (Huang, et al., Neuron, 26:443-445 (2000)). Briefly, neurons were fixed with 4% paraformaldehyde and 4% sucrose in PBS for 20 min, and permeabilized by incubation in PBS containing 1% BSA and 0.1% Triton X-100 for 30 min at room temperature. After washing, neurons were incubated in the buffer containing antibodies against phospho-ErbB4 (1:200), GAD65 (1:200), or both for 1 hr at room temperature. Brain sections (20 mm) were fixed with 10% formaldehyde and blocked in 5% BSA/1% normal goat serum (Ren, et al., Nat Neurosci, 7:1204-1212 (2004)). Sections were incubated overnight at 4° C. in PBS containing rabbit anti-ErbB4 with or without anti-GAD65 or VGAT. Fluorochrome-conjugated secondary antibodies were used to visualize the immunoreactivity with a confocal microscope.

Results

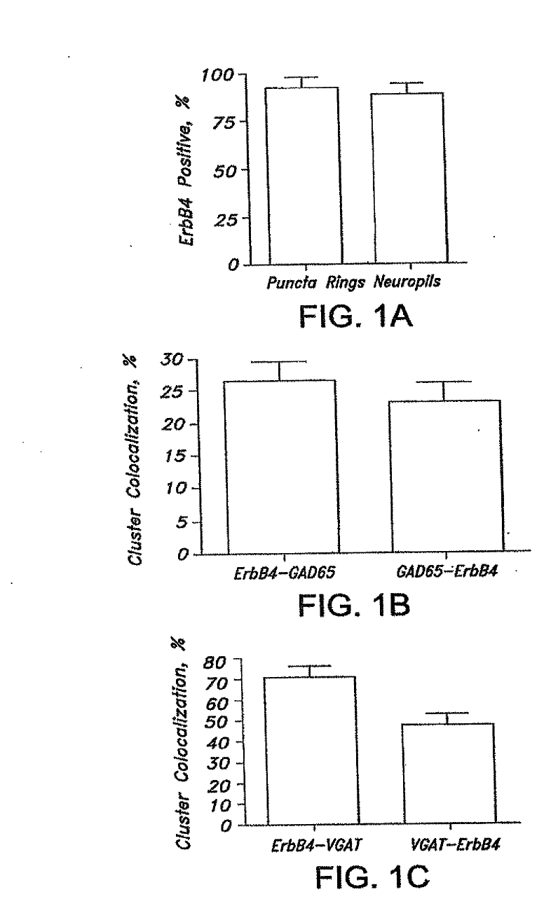

ErbB4 transcripts were expressed throughout cortical layers 2-6b (Lai and Lemke, Neuron, 6:691-704 (1991); Yau, et al., Cereb Cortex, 13:252-264 (2003)). In addition, ErbB4 transcripts were identified at high levels in the medial habenula, the reticular nucleus of the thalamus and in the intercalated masses of the amygdala. These observations are consistent with the notion that ErbB4 is expressed in interneruons. In agreement, ErbB4 was shown to be present in GAD-positive neurons isolated from the hippocampus (Huang, et al., Neuron, 26:443-455 (2000)). To determine in vivo subcellular localization of ErbB4 in GAD-positive neurons, prefrontal sections of GIN (GFP-expressing Inhibitory Neurons) mice were stained. They express GFP under the control of the gad1 promoter that directs specific expression in GABA interneurons, especially those that are somatostatin positive, in the hippocampus (Oliva, et al., J Neurosci, 20:3354-3368 (2000)). Presynaptic terminals of GABAergic neurons appear as discrete puncta rings in the prefrontal cortex, surrounding soma of postsynaptic neurons in cortical layers II-VI (Pillai-Nair, et al., J Neurosci, 25:4659-4671 (2005)). The anti-ErbB4 antibody 0618 and sc-283 specifically recognized ErbB4 because their immunoreactivity was diminished in ErbB4 mutant mice. ErbB4 was detected in puncta rings and neuropils, colocalizing with GFP. Quantitatively, about 90% of puncta rings and neuropils in the prefrontal cortex expressed ErbB4 (FIG. 1A). These results suggest that ErbB4 is present at terminals of GABAergic neurons including somatostatin neurons. To test this hypothesis further, we determined whether ErbB4 colocalizes with GAD65 and vesicular GABA transporter (VGAT), both well-characterized markers of GABAergic terminals (Tafoya, et al., J Neurosci, 26:7826-7838 (2006)). The ErbB4 immunoreactivity co-localized with GAD65 and VGAT in puncta-ring like structures (FIGS. 1B and 1C). 32% of GAD-65 clusters and 59% of VGAT clusters were ErbB4-positive, suggesting ErbB4 localization at specific subsets of GABA terminals (FIGS. 1B and 1C). On the other hand, 32% and 49% of ErbB4 clusters colocalized with GAD-65 and VGAT, respectively, in agreement with the notion that ErbB4 is also localized at non-GABAergic synapses (Huang, et al., Neuron, 26:443-455 (2000)). Taken together, these results indicate that ErbB4 is present at groups of presynaptic terminals of GABAergic neurons in the cerebral cortex.

### Example 2

### Increase in Depolarization-Evoked GABA Release by NRG1

Materials and Methods

Electrophysiological Recordings in Slices

Transverse prefrontal cortical slices (0.3 mm) were prepared from P28-P36 mice using a Vibroslice (Leica VT 1000S) in the ice-cold solution, which contained 2.5 mM KCl, 1.25 mM NaH₂PO4, 10 mM MgSO₄, 0.5 mMCaCl₂, 26 mM NaHCO₃, 10 mM glucose, and 230 mM sucrose. Slices were allowed to recover for at least 2 hr in ACSF (1 hr at 34° C. followed by 1 hr at 22° C.) in a solution containing 126 mM NaCl, 2.5 mM KCl, 1.25 mM NaH₂PO₄, 2 mM MgSO₄, 2 mM CaCl₂, 26 mM NaHCO₃, and 10 mM glucose. Slices were placed in the recording chamber and superfused (1.5 ml/min) with ACSF at 34° C. All solutions were saturated with 95% O₂/5% CO₂. Neurons were visualized with an IR-sensitive CCD camera with a 403 water-immersion lens (Zeiss, Axioskop2 Fsplus) and recorded using whole-cell voltage-clamp techniques (MultiClamp 700B Amplifier, Digidata 1320A analog-to-digital converter) and pClamp 9.2 software (Axon Instruments). Glass pipettes were filled with the solution containing 125 mM Cs-gluconate, 10 mM CsCl, 1 mM MgCl₂, 10 mM HEPES, 1 mM EGTA, 0.1 mM CaCl₂, 10 mM sodium phosphocreatine, 4 mM Mg-ATP, 0.3 mM GTP, 0.2 mM leupeptin, and 5 mM lidocaine N-ethylchloride (QX314) (pH 7.2, with the osmolarity adjusted to 280 mOsm with sucrose). The resistance of pipettes was 2-3 MΩ. For mIPSC recording, QX314 was omitted in the pipette filling solution, whereas 1 mM TTX was included in the superfusing solution. eIPSCs were generated with a two-concentric bipolar stimulating electrode (25 mm pole separation; FHC, ME) positioned about 100 mm from the neuron under recording. Single or paired pulses of 0.2 ms were delivered at 0.1 Hz and synchronized using a Mater-8 stimulator (A.M.P.I). The holding potential for both mIPSCs and eIPSCs was −65 mV. All experiments were done at 34° C. in the presence of 6-cyano-7-nitroquinoxaline-2,3-dione (CNQX, 10 mM) and AP-5 (50 mM) to block AMPA/NMDA receptors. Data were collected when series resistance fluctuated within 15% of initial values (8-15 MU), they were filtered at 2 kHz, and they were sampled at 10 kHz.

Results

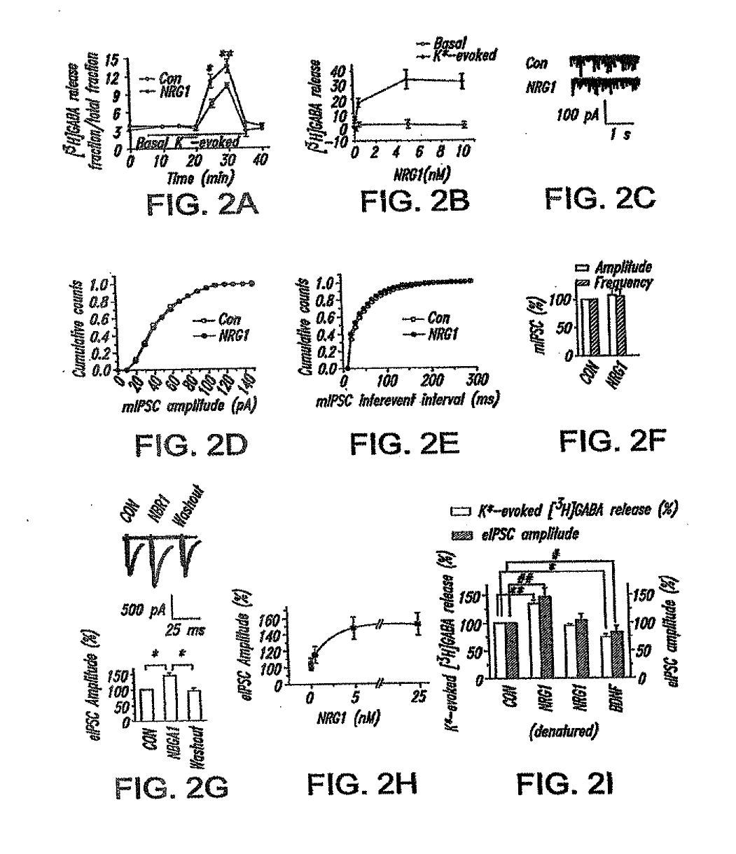

NRG1 may regulate GABAergic neurotransmission. To test this hypothesis, the effects of NRG1 on GABA release in cortical slices were determined by both biochemical and electrophysiological approaches. Basal [³H]GABA release was low, at a rate of 3.75±0.35% (n=8) of total radioactivity per 10 min (FIG. 2A). Treatment of slices with 20 mM KCl, a condition known to depolarize neurons, increased [³H]GABA release by 2.5-3.5 folds within 10 min (FIG. 2A). NRG1 had no effect on basal [³H]GABA release; by contrast, it increased depolarization-evoked GABA release in a dose-dependent manner (FIG. 2B). This effect was not inhibited by antagonists of glutamate receptors, suggesting that the increase in GABA release does not require glutamatergic signaling. To demonstrate that NRG1 regulates physiological function of GABA transmission, inhibitory postsynaptic currents (IPSCs) were recorded from prefrontal cortical slices. As shown in FIG. 2C-2F, NRG1 did not appear to affect the frequency, amplitude, and decay times of miniature IPSCs (mIPSCs) that were blockable by bicuculline, a GABA receptor antagonist. These results are in agreement with observations above that basal GABA release was not affected. By contrast, as shown in FIG. 2G, it enhanced evoked IPSCs (eIPSCs) that were sensitive to bicuculline. The increase in eIPSCs had a similar dose-response curve to evoked [³H]GABA release (FIG. 2H) and was abolished when NRG1 was heat-denatured (FIG. 2I). Furthermore, the NRG regulation remained unchanged in the presence of antagonists of metabotropic glutamate receptors, cholinergic receptors, serotonin receptors, adrenergic receptors and/or dopamine receptors. As a control, BDNF decreased depolarization-evoked GABA release and eIPSCs in cortical slices, in agreement with earlier studies (Canas, et al., Brain Res, 1016:72-78 (2004); Frerking, et al., J Neurophysiol, 80:3383-3386 (1998)). These results indicate that NRG1 increases evoked GABA release, without affecting basal release, likely via direct effect on GABAergic presynaptic terminals.

### Example 3

### NRG1 Effects on GABAergic Presynaptic Terminals

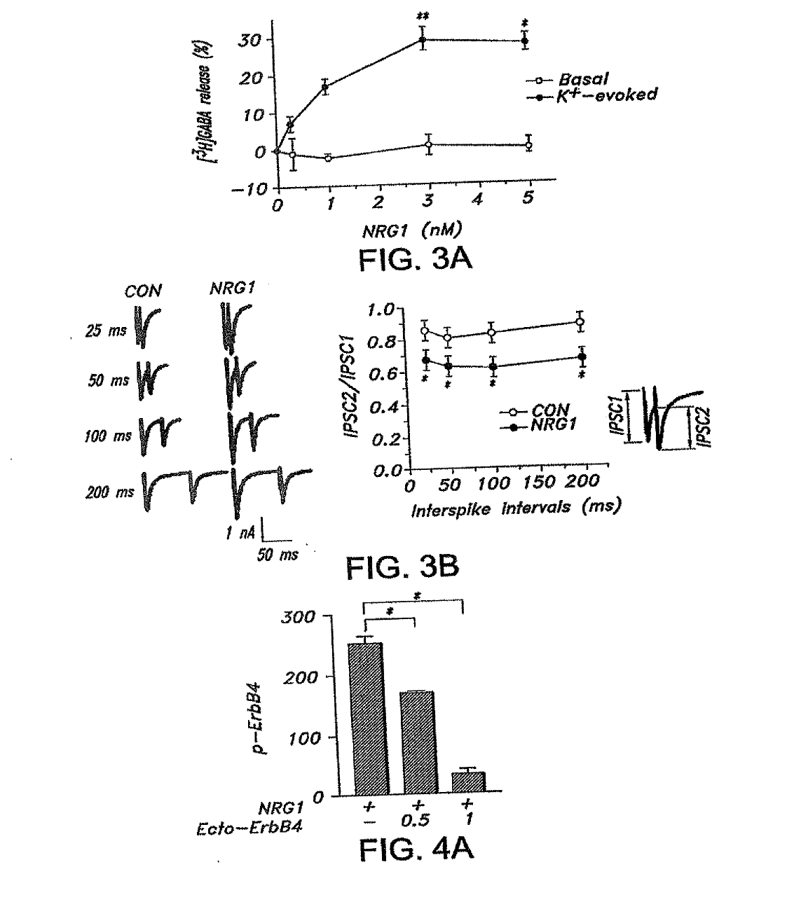

To further determine whether NRG1 regulates GABA release directly at presynaptic terminals, we performed the following two experiments. First, we investigated whether NRG1 is able to regulate [³H]GABA release from synaptosomes, free of neural circuit. As shown in FIG. 3A, NRG1 increased depolarization-evoked GABA release from synaptosomes while it had no effect on basal GABA release. Moreover, this effect was concentration-dependent, with a maximal response of 28±1.5% (n=6) similar to that observed in cortical slices (FIG. 3A). Second, we characterized the paired-pulse ratios (PPRs) of control and NRG1-affected eIPSCs in response to two stimulations. At inhibitory synapses, second stimulation generates smaller eIPSC because of depletion of vesicles in the releasable pool by the first stimulation (Lambert and Wilson, J Neurophysiol, 72:121-130 (1994)). Shown in FIG. 3B (left panel) were averaged traces of eight consecutive eIPSCs induced by paired stimuli at different interpulse intervals. The PPRs at 25 ms intervals were reduced from 0.86±0.07 in control to 0.68±0.05 in NRG1-treated slices (n=6, P<0.01). The reduction in PPRs was not recovered even at 200 ms intervals. The depression effect of NRG1 on the amplitudes of the second eIPSCs provides further evidence that NRG1 regulates evoked GABA release by a presynaptic mechanism. In addition, these results also suggest that NRG1 may increase the probability of GABA release in response to depolarization.

### Example 4

### Endogenous NRG is Necessary to Maintain Activity-Dependent GABA Release

Materials and Methods

Cell Culture

Primary cortical neurons were cultured as described previously (Huang, et al., Neuron, 26:443-455 (2000)). Briefly, cerebral cortex was dissected out of Sprague-Dawley rat embryos (E18) and dissociated by gentle trituration in PBS (Cellgro). Cells were seeded on poly-L-lysine-coated 12-well plates and cultured in Neurobasal media (Gibco). Experiments were performed 14 days after seeding (DIV14). C2C12 cells were obtained from E. S. Ralston (NIH) and cultured as previously described (Si, et al., J Biol Chem, 271:19752-19759 (1996)). To generate ecto-ErbB4, HEK293 cells were cotransfected with pC4-B4Ex/Fc, which expresses the entire ectodomain fused with the Fc fragment, and pEGFP-C1, which contains the neomycin resistance gene at a ratio of 10:1. Cells resistant to G418 (0.4 mg/ml) were cloned. Cells were cultured in 2% low Ig fetal bovine serum to collect condition medium. Ecto-ErbB4 was purified by chromatography using HiTrap protein G beads (Amersham).

Results

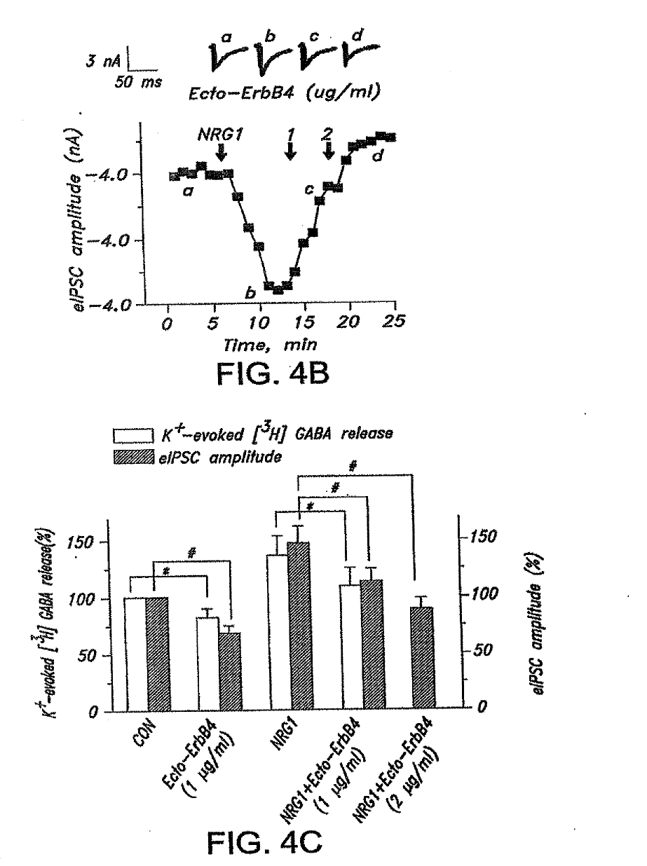

NRG1 is expressed in various regions in the brain (Law, et al., Neuroscience, 127:125-136 (2004)). NRG1-type I/II transcripts were detected prominently in cortical layer 6b and at lower levels in layers 2 and 3. In comparison, NRG1-type III transcripts were primarily detected in cortical layer 5. Hybridization of NRG1-type I/II was also observed in the reticular nucleus of the thalamus and in cholinergic interneurons in the globus pallidus. NRG1 type III was expressed in the reticular nucleus of the thalamus. Both NRG1 isoforms were also observed in the piriform cortex and throughout the hippocampus. Notably, the distinct isoforms of NRG1 appear to be expressed in a laminar-specific, and largely non-overlapping manner in the cortex. These observations indicate that NRG1 is available in various areas in the brain including the cerebral cortex. To determine whether endogenous NRG1 regulates GABA release, we generated ecto-ErbB4 that contains the entire extracellular region of ErbB4 fused to the FC fragment. Ecto-ErbB4 binds to and thus prevents NRG from interacting with ErbB receptor kinases. As shown in FIG. 4A, treatment with ecto-ErbB4 inhibited NRG1 activation of ErbB4 in GAD-positive neurons. Such treatment blocked NRG1 potentiation of eIPSCs in a dose-dependent manner (FIGS. 4B and 4C), demonstrating the neutralizing ability of ecto-ErbB4. NRG1-enhanced evoked GABA release was also inhibited by ecto-ErbB4 (FIG. 4C). Remarkably, treatment with ecto-ErbB4 alone reduced both evoked GABA release and eIPSCs in the absence of exogenous NRG1 (FIG. 4C). These observations and results from studies of inhibitors of ErbB4 suggest a role of endogenous NRG in regulating evoked GABA release.

### Example 5

### ErbB4 is Necessary for NRG1-Enhancement of Evoked GABA Release

Materials and Methods

Immunoprecipitation and Western Blotting

Immunoprecipitation was carried out as previously described (Huang, et al., Neuron, 26:443-455 (2000)). Briefly, cell lysates (1 mg of protein) were incubated with indicated antibodies (1-2 mg) at 4° C. for 1 hr with constant rocking in 1 ml of the modified RIPA buffer (50 mM Tris-HCl [pH 7.4], 150 mM NaCl, 1% NP-40, 0.25% sodium-deoxycholate, 1 mM PMSF, 1 mM EDTA, 1 mg/ml aprotinin, leupeptin, and pepstatin protease inhibitors). Samples were then incubated at 4° C. for 1 hr with agarose beads (1:1 slurry, 50 ml) conjugated with protein A (for rabbit antibodies) or G (for mouse antibodies). Bound proteins were resolved by SDS-PAGE and transferred to nitrocellulose membrane, which was blocked with TBS containing 5% nonfat dry milk and 0.05% Tween 20 for 1 hr. The membrane was then incubated overnight at 4° C. with primary antibodies and developed by horseradish peroxidase-conjugated secondary antibodies and enhanced chemiluminescence system (Amersham Pharmacia).

Results

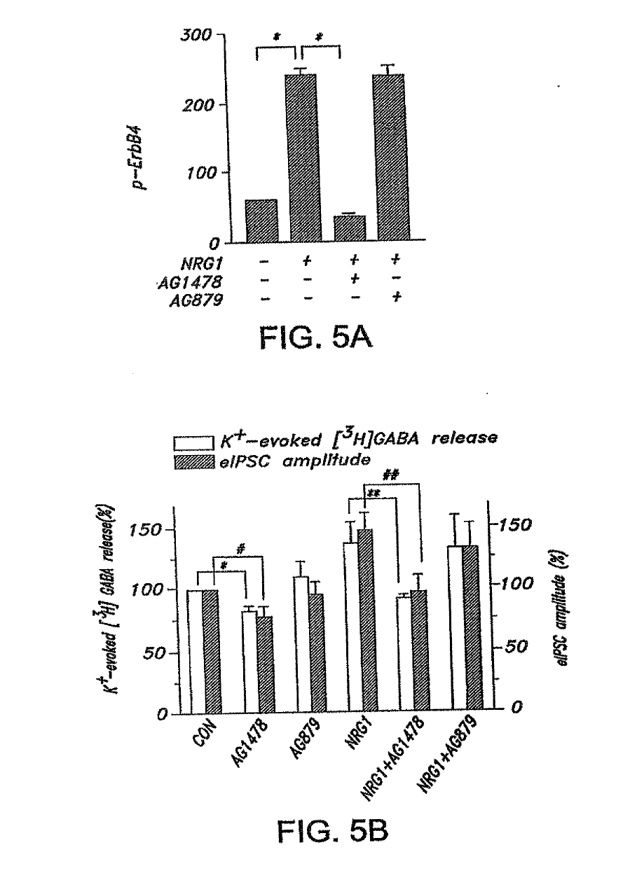

Of the three ErbB kinases, ErbB2 and ErbB4, but not ErbB3, are catalytically active (Citri and Yarden, Nat Rev Mol Cell Biol, 7:505-516 (2006)). To determine which ErbB is involved in NRG1 regulation of evoked GABA release, cortical neurons were treated with AG879 and AG1478, specific inhibitors of ErbB2 and ErbB4, respectively (Fukazawa, et al., J Mol Cell Cardiol, 35:1473-1479 (2003)). Cell culture is described in Example 4. ErbB4 tyrosine phosphorylation in response to NRG1 was blocked in neurons pretreated with AG1478, but not AG879 (FIG. 5A). Treatment with AG1478 prevented NRG1 from increasing evoked GABA release and eIPSCs in cortical slices (FIG. 5B). These results suggest a role of ErbB4 in NRG1 regulation of GABAergic transmission. As observed with ecto-ErbB4, AG1478 alone decreased depolarization-evoked [³H]GABA release and eIPSCs (FIG. 5B), providing further evidence that endogenous NRG activity may be necessary to maintain GABA release elicited by neuronal activation. As control, treatment with AG879 had no detectable effect on evoked GABA release and eIPSCs in the presence or absence of exogenous NRG1 (FIG. 5B). Taken together, these observations demonstrate that activation of ErbB4, but not ErbB2, is required for NRG1's effect.

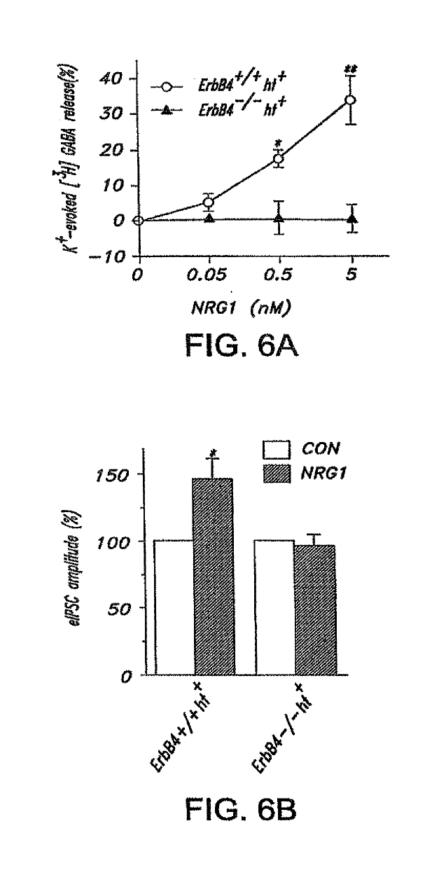

To investigate the involvement of ErbB4 further, evoked GABA release in ErbB4 mutant mice was characterized. ErbB4 null mutant mice die around embryonic day 11. The embryonic lethality can be genetically rescued by expressing ErbB4 under a cardiac-specific myosin promoter (Tidcombe, et al., Proc Natl Acad Sci USA, 100:8281-8286 (2003)). This line of mice (ErbB4−/−ht⁺), however, do not express ErbB4 in the brain or other non-cardiac tissues (data not shown). Ablation of the ErbB4 gene had no effect on basal and depolarization-evoked [³H]GABA release (FIG. 6A). However, unlike control slices, NRG1 was unable to increase evoked [³H]GABA release and eIPSCs in ErbB4−/−ht⁺ slices (FIGS. 6A and 6B). These observations identify an important role of ErbB4 in NRG1 regulation of evoked GABA release.

The data presented herein provide evidence that ErbB4 is present at GABAergic terminals in the prefrontal cortex. The identification of the subtype or subtypes of GABA interneurons that express ErbB4 will require further investigation. Interestingly, ErbB4 colocalizes with GAD-GFP in GIN mice. An earlier study demonstrated that hippocampal GAD-GFP-labeled neurons of these mice are mostly somatostatin positive (Oliva, et al., J Neurosci, 20:3354-3368 (2000)). Whether GFP-labeled neurons in the prefrontal cortex are somatostatin positive was not characterized in detail. Nevertheless, the data show that NRG1 activates ErbB4 and regulates GABAergic transmission. This trophic factor has no effect on basal GABA release but increases GABA release evoked by neuronal activation. Because glutamatergic neurotransmission can be regulated by NRG1 (Gu, et al., J Neurosci, 25:4974-4984 (2005); Li, et al., Neuron, (in press) (2007)) and because glutamatergic activity is known to increase GABAergic transmission (Belan and Kostyuk, Pflugers Arch, 444:26-37 (2002)), it is possible that NRG1 regulation of evoked GABA release may be mediated by a glutamatergic mechanism.

The results provided herein, however, suggest otherwise; NRG1 enhancement of evoked [³H]GABA release was not attenuated by inhibitors of NMDA and AMPA receptors. Moreover, NRG1 enhanced eIPSCs in the presence of these inhibitors. Therefore, it is likely that NRG1 regulates GABA release by directly activating ErbB4 receptors on presynaptic terminals. The presence of ErbB4 in GAD-GFP-positive puncta-ring-like structures and the colocalization with GAD65 and VGAT provide anatomical evidence in support of this notion. Moreover, NRG1 was able to increase depolarization-evoked GABA release from synaptosomes that were free of interneural network, suggesting that the regulatory machinery for NRG1 was present in presynaptic terminals. Furthermore, NRG1 decreases PPRs of eIPSCs in response to two consecutive stimulations, suggesting that it may facilitate vesicle release evoked by neuronal activation of interneurons.

The data provided here provides evidence that endogenous NRG1 plays a role in maintaining evoked GABA release. First, treatment with ecto-ErbB4 alone attenuated evoked GABA release, presumably by neutralizing endogenous NRG1. Second, inhibition of ErbB4 reduced evoked GABA release in the absence of exogenous NRG1. In light of the fact that interneuron activity in vivo could be high (Mountcastle, et al., J Neurophysiol, 32:452-484 (1969)), it is likely that NRG1 plays an important role in controlling neuronal activity in the brain. These data are consistent with expression of NRG1 by cortical pyramidal neurons and ErbB4 by interneurons. While ErbB4 is expressed in interneurons throughout the cortex, distinct isoforms of NRG1 appear to be expressed in a lamina-specific and largely non-overlapping manner in the cortex. The readily available NRG1 may maintain basal activity-dependent GABAergic transmission. Interestingly, NRG1 or ErbB4 heterozygotes show hyperactivity in an open field (Gerlai, et al., Behav Brain Res, 109:219-227 (2000); Stefansson, et al., Am J Hum Genet, 71:877-892 (2002)).

Statistical Analysis

Data were presented as mean±SEM of three or more independent experiments. For multiple group comparisons, statistical differences were calculated by one-way ANOVA followed by Dunnett's test. For comparison of means from the same group of cells, Student's paired t test was used. mIPSCs were analyzed by the Kolmogorov-Smirnov (K-S) test. Values of p<0.05 were considered significant.

### Example 6

### NRG1 Inhibits Firing Rate of Pyramidal Neurons in the PFC

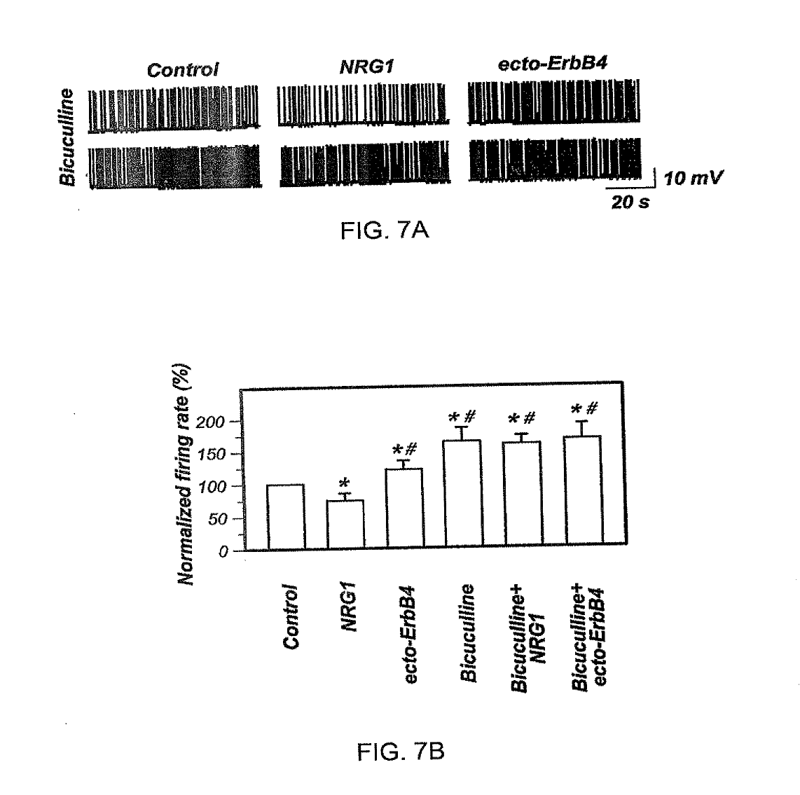

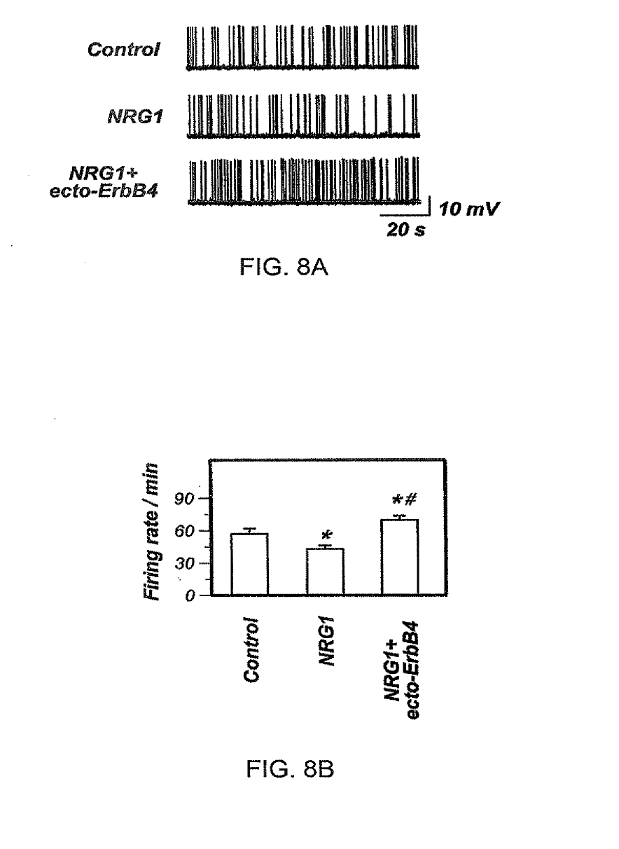

To determine whether NRG1 regulates the activity of PFC pyramidal neurons, spontaneous firing rates were recorded extracellularly in a loose-patch cell-attached configuration. Pyramidal-like neurons with triangular shaped soma and prominent apical dendrites in layers II-V of coronal PFC sections were visually identified by infrared-differential interference contrast optics. The spontaneous firing rates were 55.9±7.8/min (n=7). Bath 5 nM NRG1 reduced the spontaneous firing rates within 5 min of application (P<0.05, n=7; FIGS. 7A and B), suggesting that NRG1 could regulate PFC pyramidal neuron activity. This effect was blocked by 1 μg/ml ecto-ErbB4, a NRG1 neutralizing peptide (Woo, et al., Neuron, 54:599-610 (2007)) (FIGS. 8A and B). Interestingly, ecto-ErbB4 alone increased firing rates of PFC pyramidal neurons in the absence of exogenous NRG1 (P<0.05) (FIGS. 7A and B), suggesting a necessary role of endogenous NRG1 in maintaining pyramidal neuron activity.

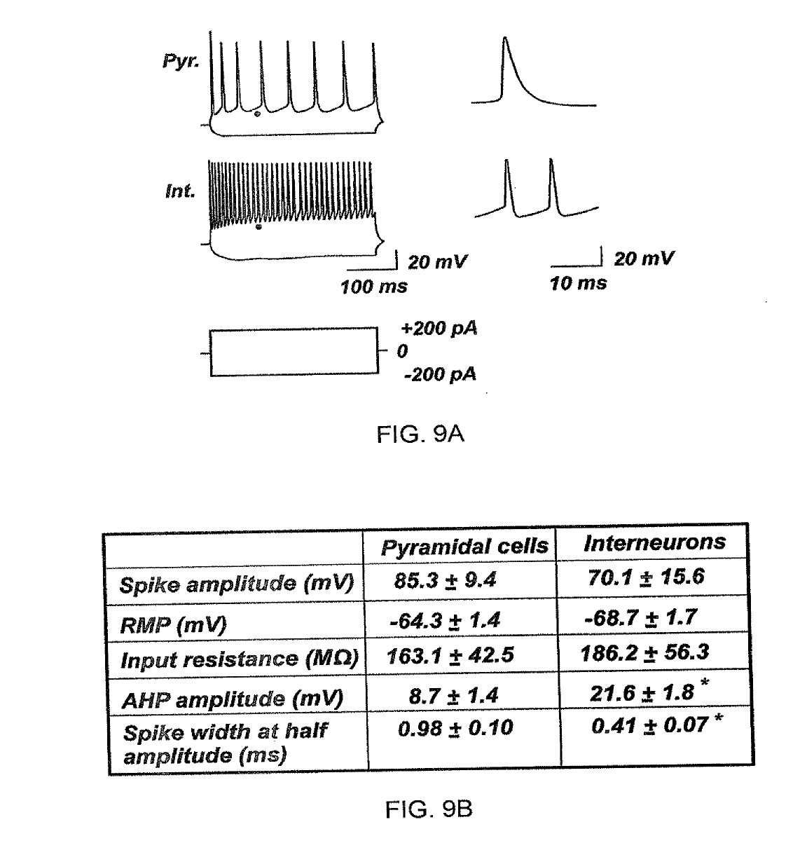

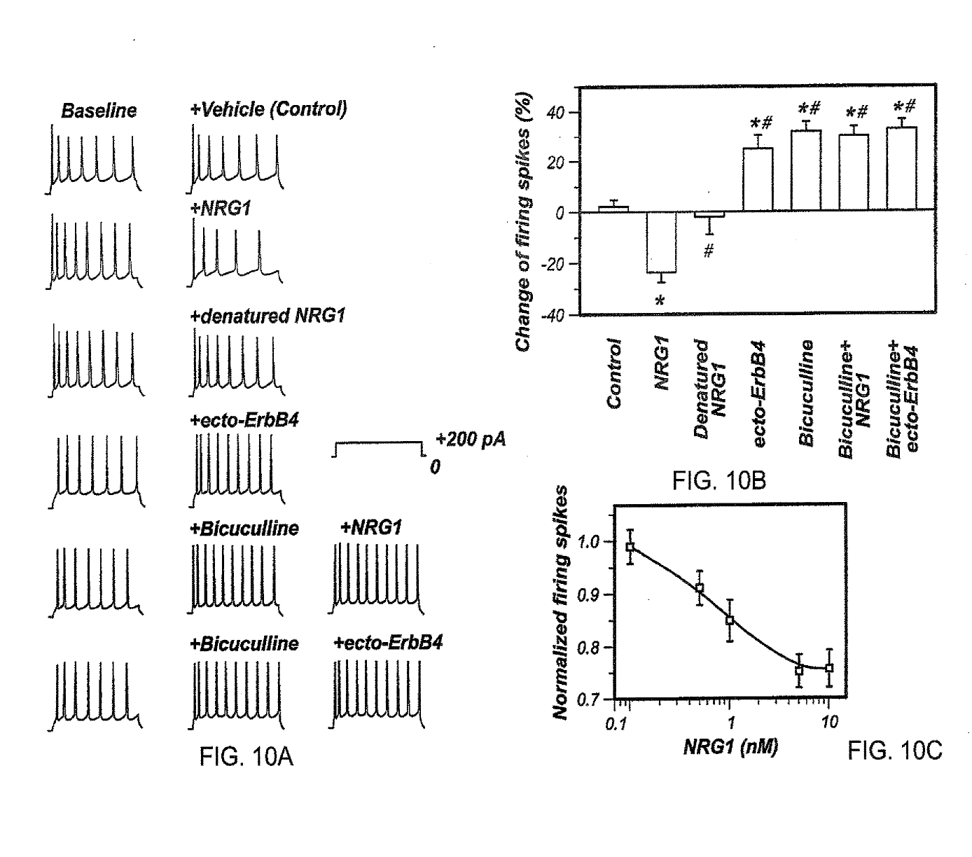

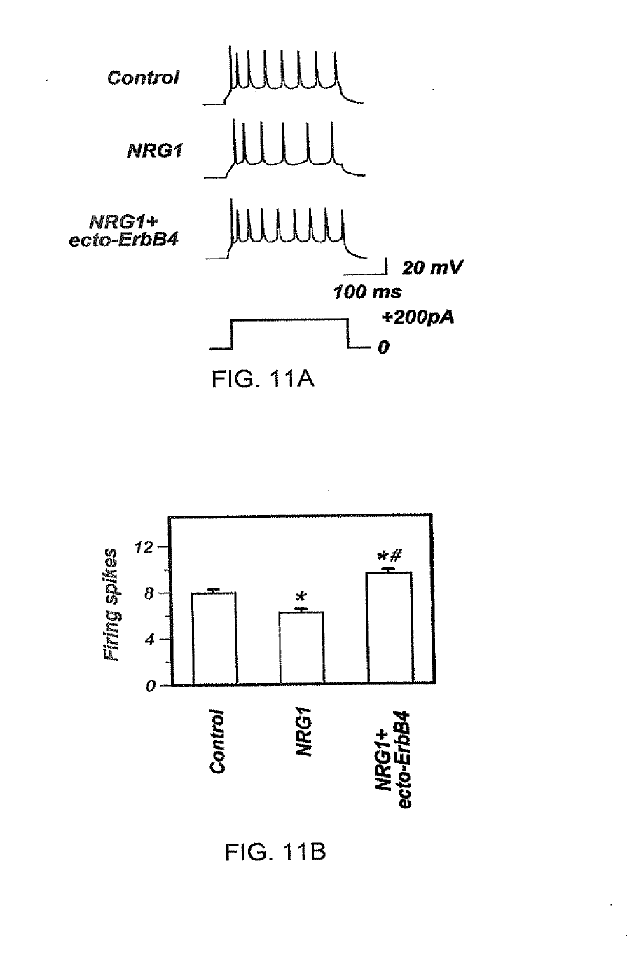

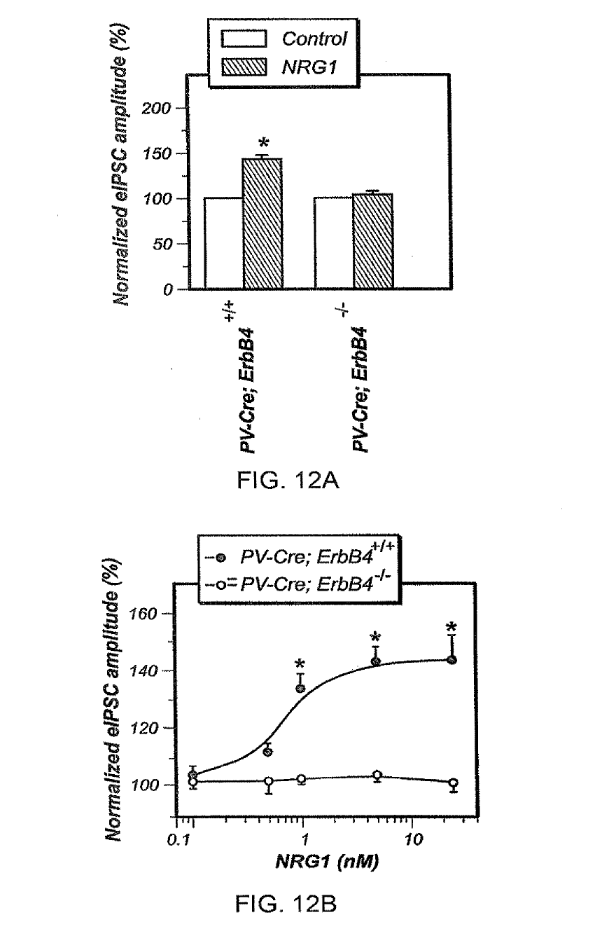

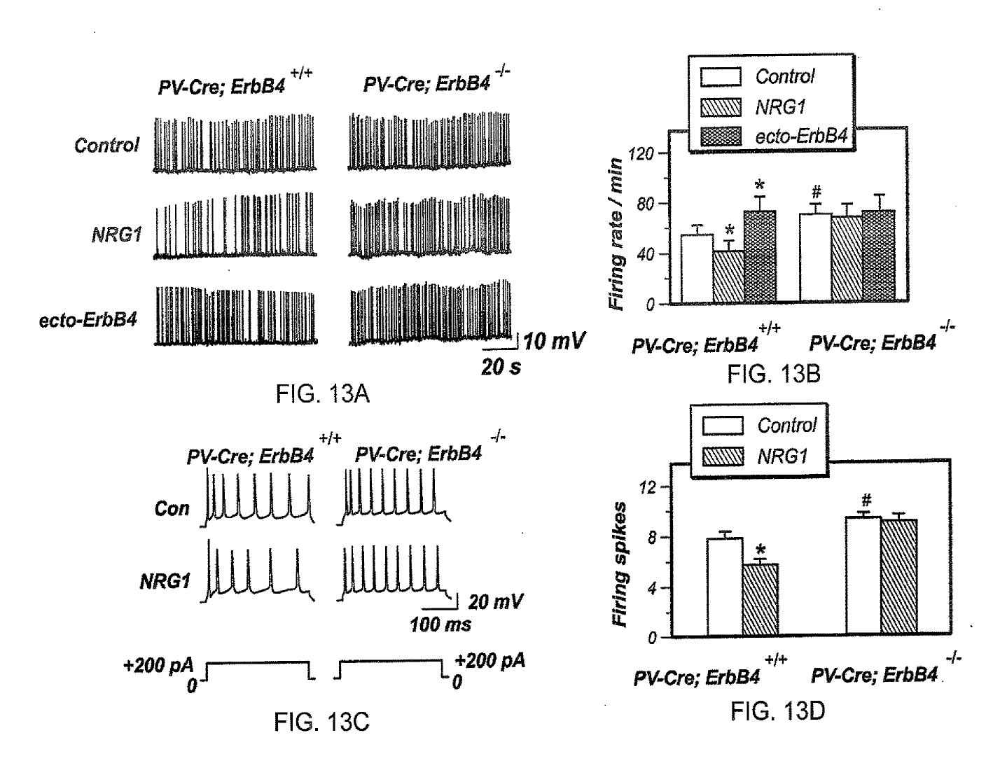

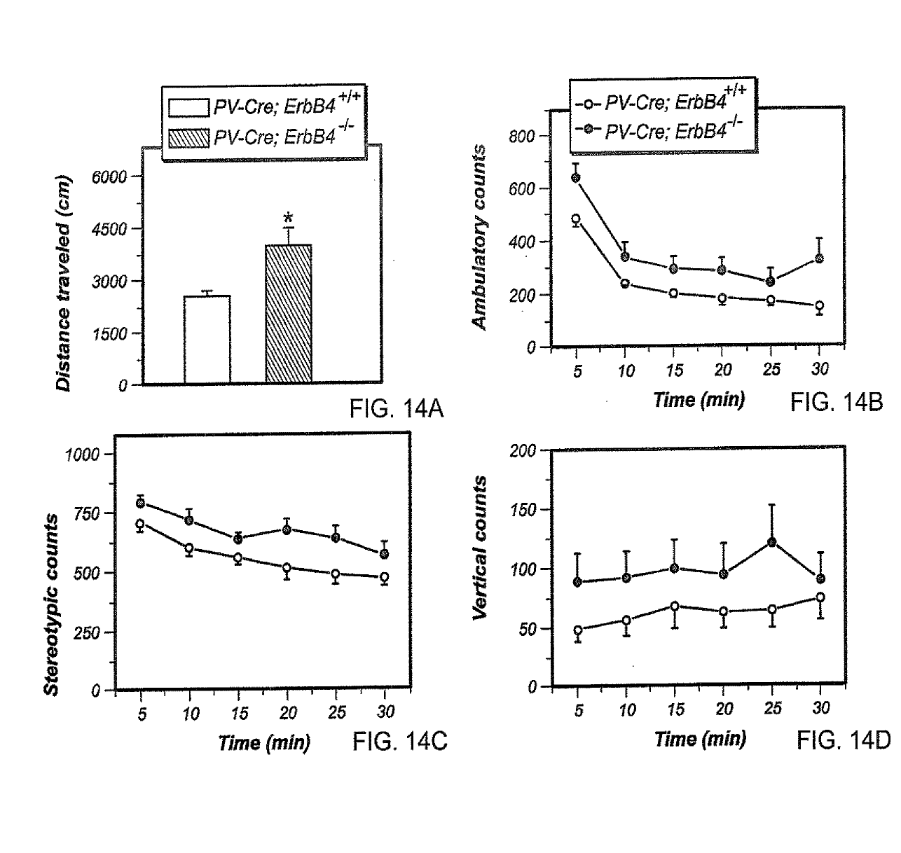

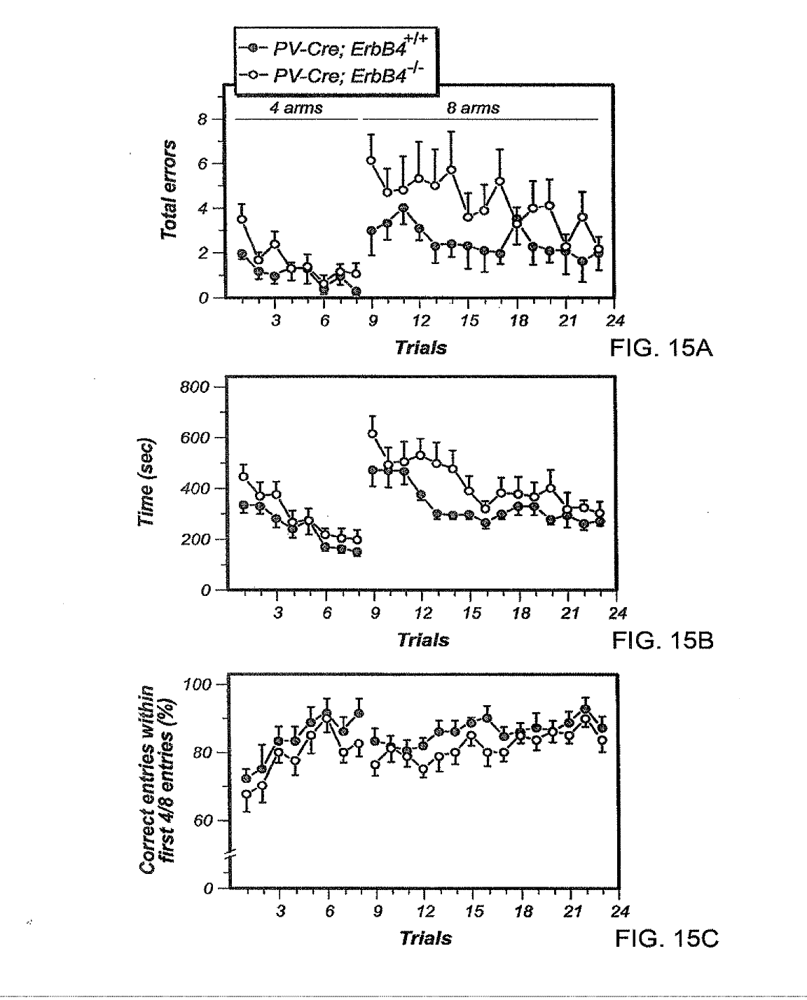

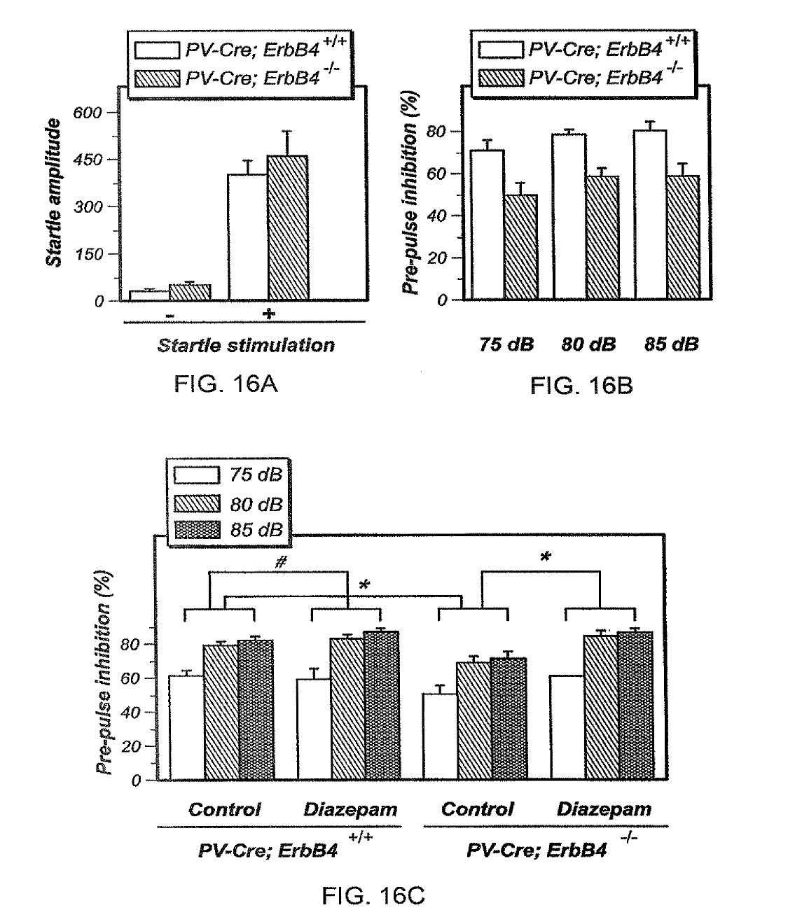

To test this idea further, action potentials of PFC layers II-V pyramidal neurons that were generated by a 200-pA suprathreshold somatic current injection in a whole-cell patch-clamping configuration were recorded. Pyramidal neurons exhibited a characteristic spiking adaptation (FIGS. 9A and B) (Markram, et al., Nat Rev Neurosci, 5:793-807 (2004)) and could be costained with calcium/calmodulin-dependent protein kinase II (CaMKII), a marker of pyramidal neurons. The evoked firings of pyramidal neurons also differed from those of interneurons in the after-hyperpolarization amplitude and spike width at half amplitude (FIGS. 9A and B) (Markram, et al., Nat Rev Neurosci, 5:793-807 (2004)).

In agreement with the results of loose-patch recording, bath application of NRG1 decreased the number of action potentials of PFC layers II-V pyramidal neurons (FIGS. 10A and B). During 300 ms of current injection, action potential numbers were reduced from 8.3 0.58 in control to 6.2±0.32 in slices treated with 5 nM NRG1 (n=9, P<0.05). The inhibitory effect was evident within 5 min of application and disappeared ˜5 min after removal of NRG1. This effect did not appear to be non-specific because it was abolished by heat inactivation of NRG1 (FIGS. 10A and B) and was blocked by the neutralizing peptide ecto-ErbB4 (FIGS. 11A and B). Moreover, the effect was concentration-dependent with an IC50 value similar to that on GABA release (FIG. 10C) (Woo, et al, Neuron, 54:599-610 (2007). Spike generation was inhibited by 24.8±3.5% at maximal concentrations.

These results demonstrate that NRG1 was able to inhibit the activity of pyramidal neurons in the PFC. As observed in the loose-patch studies, treatment with 1 μg/ml ecto-ErbB4 alone increased the number of action potentials (from 7.8±0.72 in control to 9.4±0.93 in slices treated with ecto-ErbB4, n=9, P<0.05) (FIGS. 10A and B), suggesting that the evoked spike frequency of pyramidal neurons is regulated by endogenous NRG.

NRG1 inhibits the activity of pyramidal neurons in the PFC. Both spontaneous firing rates and the frequency of evoked action potentials in pyramidal neurons were reduced by NRG1, but increased by the neutralizing peptide ecto-ErbB4. NRG1 enhances activity-dependent release of GABA (Woo, et al, Neuron, 54:599-610 (2007)). To investigate if the NRG1 regulation of pyramidal neuron firing requires GABA release, the effects of NRG1 on pyramidal neuron firing were studied under bicuculline, a selective antagonist of GABAA receptors. The firing rate of pyramidal neurons as measured by both loose patch and whole-cell patch techniques was increased by 20 M bicuculline (FIGS. 7A and B and FIGS. 10A and B). Remarkably, in the presence of bicuculline, NRG1 was no longer able to suppress pyramidal neuron activity, suggesting a requirement for GABAA receptor activation in this event (FIGS. 7A and B and FIGS. 10A and B). Furthermore, bicuculline blocked the effect of ecto-ErbB4 (FIGS. 7A and B and FIGS. 10A and B). Together, these results support the concept that NRG1, via increasing GABA release, regulates pyramidal neuron activity.

The inhibitory effect of NRG1 on the activity of pyramidal neurons could be mediated by decreased excitatory synaptic input and/or increased inhibitory synaptic activity. Evoked action potentials were recorded under a condition where most, if not all, of glutamatergic transmission is blocked, making it unlikely to involve excitatory synaptic activity or input. In contrast, the inhibitory effect of NRG1 on spontaneous and evoked spike generation was blocked by bicuculline (FIG. 7 and FIG. 10), indicating the involvement of GABA transmission. This idea was supported by the studies of ecto-ErbB4, a neutralizing peptide that reduces activity-dependent GABA release (Woo, et al., Neuron, 54:599-610 (2007)).

### Example 7

### ErbB4 is Critical to NRG1 Potentiation of GABA Release and Suppression of Pyramidal Neuron Activity

Materials and Methods

Western Blotting

PFC was isolated from PV-Cre;ErbB4−/− and control littlemates (PV-Cre;ErbB4+/+) and homogenized. Resulting homogenates (40 μg of protein) were subjected to western blotting analysis with antibodies against ErbB4. Equal loading was shown by immunoblotting for β-actin.

Immunostaining

PFC slices of PV-Cre;ErbB4−/− and PV-Cre;ErbB4+/+ were stained with anti-ErbB4 and PV antibody. Immunoactivity was visualized by Alexa 488- and Alexa 594-conjugated secondary antibodies, respectively. Slices were also stained with DAPI to indicate nuclei.

Mice

ErbB4lox/lox and PV-Cre mice were described previously (Garcia-Rivello, et al. Am. J. Physiol Heart Circ Physiol. 289:H1153-1160, Arber, et al., Cell, 101:485-98), Hippenmeyer, et al., PLoS Biol. 3:e159 (2005)). PVCre;ErbB4−/− and control mice were housed in a room with a 12-hr light/dark cycle with free access to food and water ad libitum unless otherwise indicated. Experiments with animals were approved by IACUC of the MCG.

Results

ErbB4 was ablated specifically in parvalbumin (PV)-positive neurons in PV-Cre;ErbB4−/− mice where expression of Cre is not active until postnatal day (Del Rio, et al., Brain Res Dev Brain Res, 81:247-259 (1994); Hof, et al., J Chem Neuroanat, 16:77-116 (1999)), a time when the cortical lamination is nearly achieved (Hof, et al., J Chem Neuroanat, 16:77-116 (1999); Finlay and Darlington, Science, 268:1578-1584 (1995)). NRG1 regulation of evoked GABA release is abolished in PFC slices from ErbB4−/− mice, suggesting that it requires ErbB4 (Woo, et al., Neuron, 54:599-610 (2007)). ErbB4 is highly expressed in PV-positive interneurons (Yau, et al., Cereb Cortex, 13:252-264 (2003); Woo, et al., Neuron, 54:599-610 (2007); Vullhorst, et al., J Neurosci, 29:12255-64 (2009)), which have been implicated in controlling the output of pyramidal neurons (Markram, et al., Nat Rev Neurosci, 5:793-807 (2004); McBain and Fisahn, Nat Rev Neurosci, 2:11-23 (2001)). If the NRG1-mediated reduction of the firing rate of pyramidal neurons results from an increase in GABAergic transmission, the effect should require ErbB4 in PV-positive interneurons. To test this idea, ErbB4 expression was specifically ablated in PV-positive interneurons by crossing ErbB4lox/lox mice (Garcia-Rivello, et al., Am J Physiol Heart Circ Physiol, 289:H1153-1160 (2005)) with PV-Cre mice (Arber, et al., Cell, 101:485-498 (2000); Hippenmeyer, et al., PLoS Biol, 3:e159 (2005)). Western blotting analysis indicated that ErbB4 was reduced but not abolished in the PFC of PV-Cre;ErbB4−/− mice. This result was not unexpected because ErbB4 has been shown to be expressed by other neurons including glutamatergic neurons (Garcia, et al., Proc Natl Acad Sci USA, 97:3596-3601 (2000); Huang, et al., Neuron, 26:443-455 (2000); Ma, et al., J Neurosci, 23:3164-3175 (2003); Li, et al., J Biol Chem, 278:35702-35709 (2003)).

To determine the extent of ErbB4 deletion in PV-positive neurons, PFC sections were co-stained with antibodies against PV and ErbB4. ErbB4 was detectable in almost all of PV-positive neurons and in neurons that were not positive for PV in control littermates, in agreement with previous studies (Yau, et al., Cereb Cortex, 13:252-264 (2003); Vullhorst, et al., J Neurosci, 29:12255-12264 (2009); Fisahn, et al., Cereb Cortex, 19:612-618 (2009); Neddens and Buonanno, Hippocampus, (Epub ahead of print) (2009)). In contrast, ErbB4 immunoreactivity was abolished in PV-positive, but not PV-negative neurons in PV-Cre;ErbB4−/− slices. These results demonstrated the specific loss of ErbB4 in PV-positive neurons.

Next, the effect of the PV-specific ErbB4 knockout on evoked inhibitory postsynaptic current (eIPSC) amplitudes was investigated. As observed previously (Woo, et al., Neuron, 54:599-610 (2007)), 5 nM NRG1 increased eIPSC amplitudes (by 43.2±5.1%) in PV-Cre;ErbB4+/+ PFC slices within 5 min (FIG. 12A). Remarkably, this effect was abolished in PFC slices from age-matched PV-Cre;ErbB4−/− mice. NRG1 showed little, if any, effect on eIPSC amplitudes in mutant slices even at a concentration 5-fold higher than that eliciting a maximal response in control slices (FIG. 12B). These results indicate a critical role for NRG1/ErbB4 signaling in PV-positive neurons to regulate GABAergic transmission. In addition, both the spontaneous firing rate in loose-patch recordings (FIGS. 13A and B; n=12 for both genotypes) and the spikes evoked by a 200-pA current injection in whole-cell recordings (FIGS. 13C and D; n=9 for both genotypes) were increased in PV-Cre;ErbB4−/− slices in comparison to those from PV-Cre;ErbB4+/+ mice (P<0.05), indicating that the loss of ErbB4 in PV-positive neurons increases the activity of pyramidal neurons. Importantly, unlike control slices where NRG1 had an inhibitory effect, NRG1 was unable to decrease the firing rate of pyramidal neurons in PV-Cre;ErbB4−/− slices (FIG. 13A-D). Similarly, the ability of ecto-ErbB4 to increase the rate of firing was also lost in PV-Cre;ErbB4−/− mice (FIGS. 13A and B). These results suggest that regulation of GABAergic transmission by both exogenous and endogenous NRG1 and the subsequent inhibition of pyramidal neuron firing require ErbB4 in PV-positive inhibitory interneurons.

GABAergic interneurons are a heterogeneous group of neuronal cells with distinct functions (Markram, et al., Nat Rev Neurosci, 5:793-807 (2004)). Basket cells synapse onto the somata and proximal dendrites of pyramidal neurons whereas chandelier cells preferentially target their axon initial segments. These interneurons regulate the output of pyramidal neurons by affecting the generation and timing of action potentials (Markram, et al., Nat Rev Neurosci, 5:793-807 (2004); McBain and Fisahn, Nat Rev Neurosci, 2:11-23 (2001)). Both basket and chandelier cells express calcium-binding proteins PV (Markram, et al., Nat Rev Neurosci, 5:793-807 (2004); Lewis, et al., Nat Rev Neurosci, 6:312-324 (2005)). Interestingly, the potentiation effect of NRG1 on GABAergic transmission was abolished in PV-Cre;ErbB4−/− mice (FIG. 12A-B) and NRG1 was no longer able to inhibit spontaneous firing rates and the frequency of evoked action potentials in pyramidal neurons (FIG. 13). These observations provide convincing evidence that PV-positive neurons are a major cellular target of NRG1/ErbB4 signaling in regulating GABAergic transmission and pyramidal neuron activity.

### Example 8

### PV-Cre;ErbB4−/− Mice are Hyperactive

Material and Methods

Behavioral analysis was carried out with 8-12 week old mice by an investigator unaware of their genotype.

Results

To gain insight into the physiological function of ErbB4 in PV-positive interneurons, PV-Cre;ErbB4−/− mice (8-12 weeks old at the start of experiments) were subjected to a series of behavioral tests in a blind manner. PVCre;ErbB4−/− mutant mice did not exhibit differences in weight, whisker number and rectal temperature in comparison with wild-type littermates, and there were no significant differences in motor coordination. First PV-Cre;ErbB4−/− mice were investigated for hyperactivity, a characteristic rodent phenotype that is thought to correspond to the psychomotor agitation of schizophrenic patients (Gainetdinov, et al., Trends Neurosci, 24:527-533 (2001); Geyer and Ellenbroek, Prog Neuropsychopharmacol Biol Psychiatry, 27:1071-1079 (2003); Arguello and Gogos, Neuron, 52:179-196 (2006)). Hyperactivity has been reported in mice heterozygous for NRG1 or ErbB4 (Stefansson, et al., Am J Hum Genet, 71:877-892 (2002); Gerlai, et al., Behav Brain Res, 109:219-227 (2000); Golub, et al., Behav Brain Res, 153:159-170 (2004); O'Tuathaigh, et al., Neuroreport, 17:79-83 (2006); O'Tuathaigh, et al., Neuroscience, 147:18-27 (2007); O'Tuathaigh, et al., Neurosci Biobehav Rev, 31:60-78 (2007)). Strikingly, PV-Cre;ErbB4−/− mice showed consistent hyperactivity in the open field test in comparison with wild type controls (FIG. 14A-D). They traveled a significantly greater distance (FIG. 14A) [n=7 and 6 for control and mutant mice, respectively; F(1,11)=3.735, P=0.017]. Ambulatory counts revealed a significant genotype effect [FIG. 14B; repeated measures, genotype F(1,11) 5.096, P=0.045], suggesting abnormally higher horizontal or locomotory activity of the mutant mice. In addition, PV-Cre;ErbB4−/− mice showed higher stereotypic activity [FIG. 14C; genotype F(1, 11)=5.237, P=0.043]. Notice that both mutant and control mice exhibited habituation of locomotory and stereotypic activity with time and the rate of habituation was not different between mutant and control [for locomotory activity: time F(5,55)=81.073, P<0.001; genotype time interaction F(5,55)=1.746, P=0.139; for stereotypic activity: time F(5,55)=16.542, P<0.001; genotype time interaction F(5,55)=1.566, P=0.185], indicating that both wild type and mutant mice were able to adapt to a novel environment. No difference was observed in vertical or rearing activity between control and mutant mice [FIG. 14D; genotype F(1, 11)=1.844, P=0.202; time F(5,55)=1.553, P=0.189; genotype time interaction F(5,55)=1.084, P=0.380]. Together these results are consistent with the idea that specific ablation of ErbB4 in PV-positive neurons increased locomotor activity and stereotypical activity.

### Example 9

### Working Memory is Impaired in PV-Cre;ErbB4−/− Mice

Working memory deficits are thought to be central to poor cognitive performance in schizophrenia and to result from GABAergic dysfunction (Lewis, et al., Nat Rev Neurosci, 6:312-324 (2005); Lewis and Gonzalez-Burgos, Nat Med, 12:1016-1022 (2006)). To examine whether the loss of ErbB4 in PV-positive interneurons resulted in cognitive deficits, PV-Cre;ErbB4−/− mice and control littermates were evaluated for their performance on an automated radial arm maze to assess changes in working memory. Food-restricted mice were trained to retrieve food pellets from the end of each arm. After the initial shaping, mice were allowed free access to either 4 arms (less difficult condition) or 8 arms (more difficult condition) where all arms were baited. The number of errors (repeated entries into a previously visited arm or omission of an arm) and the total time to retrieve all pellets were scored.

When mice were analyzed in the 4-arm test, a significant trial effect was observed in total number of errors and the total time to retrieve all pellets [n=9 and 10 for control and mutant mice, respectively; repeated measures for total errors, trial F(7, 119)=4.532, P<0.001; for total time, trial F(7, 119)=−10.532, P<0.001], but there was no difference in genotype trial interaction [for total errors, F(7,119)=0.880, P=0.525; for total time, F(7,119)=0.470, P=0.855](FIG. 15A-B). These results indicated that both control and PV-Cre;ErbB4−/− mice were able to learn to retrieve food pellets by reducing wrong entries and exploration time. Although there was no significant difference between total time mutant and control mice spent to retrieve all food pellets [repeated measures, genotype F(1, 17)=3.097, P=0.096; FIG. 15B], PV-Cre;ErbB4−/− mice showed a significantly increased number of total errors [repeated measures, genotype F(1, 17)=14.158, P=0.002; FIG. 15A], suggesting possible deficits in working memory.

Because PV-Cre;ErbB4−/− mice were hyperactive (FIG. 14A-D), the increase in total errors may have resulted from random hyperactivity. To exclude this possibility, correct entries were monitored during the first 4 entries, eliminating effects of total travel distance and time (Gerlai, et al., Behav Brain Res, 109:219-227 (2000)). Intriguingly, the percentage of correct entries within the first 4 entries was significantly lower in mutant mice in comparison with controls [repeated measures, genotype F(1, 17)=4.729, P=0.044, trial F(7, 119)=5.173, P<0.001, genotype trial interaction F(7,119)=0.121, P=0.997; FIG. 15C]. These results are in agreement with the idea of impaired working memory in PV-Cre;ErbB4−/− mice.

To test this hypothesis further, the difficulty of the working memory test was increased by using an 8-arm radial maze. Both total wrong entries and the percentage of correct entries within the first 8 entries were significantly lower from wild type control littermates [repeated measure, F(1, 17)=6.436, P=0.021 for total wrong entries; F(1, 17)=4.952, P=0.040 for percentage of correct entries within the first 8 entries; FIGS. 15A and C]. These results demonstrated that ErbB4 in PV-positive interneurons is critical for working memory, suggesting that the alteration of NRG1/ErbB4 signaling in PV-positive interneurons might contribute to the cognitive deficits in schizophrenia.

### Example 10

### Pre-Pulse Inhibition (PPI) is Attenuated in PV-Cre;ErbB4−/− Mice

Patients with schizophrenia often show deficits in pre-pulse inhibition (PPI), a common test of sensory gating that can also be performed in rodents (Gainetdinov, et al., Trends Neurosci, 24:527-533 (2001); Geyer and Ellenbroek, Prog Neuropsychopharmacol Biol Psychiatry, 27:1071-1079 (2003); Arguello and Gogos, Neuron, 52:179-196 (2006)). Reduced PPI ability is thought to contribute to schizophrenic conditions including inattention, distractibility, and cognitive deficits. Single nucleotide polymorphisms (SNPs) in the nrg-1 gene is associated with PPI deficits in schizophrenic patients (Mei and Xiong, Nat Rev Neurosci, 9:437-452 (2008); Lin, et al., Psychol Med, 35:1589-1598 (2005); Hong, et al., Biol Psychiatry, 63:17-23 (2008)) and mutation of the mouse homologue leads to reduced PPI in mice (Barros, et al., Proc Natl Acad Sci USA, 106:4507-4512 (2009); Stefansson, et al., Am J Hum Genet, 71:877-892 (2002)). A combination of startle (120 dB) and three levels of pre-pulses (75 dB, 80 dB, and 85 dB) were used.

Mutant and wild type mice produced similar startle responses to 120 dB stimuli [n=7 and 6 for control and mutant mice, respectively; F(1, 11)=1.925, P=0.539; FIG. 16A]; however, PPI was significantly lower in PV-Cre;ErbB4−/− mice in comparison with controls [repeated measures, genotype F(1, 11)=13.684, P=0.004; FIG. 16B]. It should be noted that significant pre-pulse intensity effects were also observed [repeated measures, pre-pulse intensity F(2, 22)=10.615, P=0.001; genotype pre-pulse intensity F(2, 22)=0.113, P=0.894; FIG. 16B]. These results indicated that both mutant and control mice could distinguish among each pre-pulse intensity; however, PV-Cre;ErbB4−/− mice are impaired in PPI.

To investigate whether abnormal GABAergic transmission may be a cause to behavioral deficits, PV-Cre;ErbB4−/− mice were treated with diazepam, a GABA enhancer. PPI remained deficient in the mutant mice treated with vehicle [repeated measures, genotype F(1, 20)=5.302, P=0.032; n=10 and 12 for PV-Cre;ErbB4−/− and control littermates, respectively; FIG. 16C]. 3 mg/kg diazepam seemed to have no effect on PPI after 30-min treatment in control mice [repeated measures, genotype F(1, 17)−0.330, P=0.573; n=10 and 9 for vehicle and diazepam, respectively; FIG. 16C], in consistent with previous report (Ouagazzal, et al., Psychopharmacology (Berl), 156:273-283 (2001)).

In contrast, diazepam significantly enhanced PPI in PVCre; ErbB4−/− mice [repeated measures, genotype F(1, 21)=6.157, P=0.022; n=12 and 11 for vehicle and diazepam, respectively; FIG. 16C]. These results indicated that acute administration of low-dose diazepam was able to ameliorate disrupted PPI in PV-Cre;ErbB4−/− mice, providing evidence for impaired GABAergic transmission.

NRG1 hypomorphic mice including Nrg1(?EGF)+/ and Nrg1(?TM)+/ mice were hyperactive in novel open-field and alternating-Y maze tests (Stefansson, et al., Am J Hum Genet, 71:877-892 (2002); Gerlai, et al., Behav Brain Res, 109:219-227 (2000); O'Tuathaigh, et al., Neuroreport, 17:79-83 (2006); O'Tuathaigh, et al., Neuroscience, 147:18-27 (2007); O'Tuathaigh, et al., Neurosci Biobehav Rev, 31:60-78 (2007)). Nestin-Cre;ErbB4−/− mice were more active than control at the initial stage of behavioral evaluation (Golub, et al., Behav Brain Res, 153:159-170 (2004)). Nrg1 and ErbB4 mutant mice are impaired in PPI (Barros, et al., Proc Natl Acad Sci USA, 106:4507-4512 (2009); Stefansson, et al., Am J Hum Genet, 71:877-892 (2002)). Interestingly, these schizophreniarelevant behaviors of NRG1 and ErbB4 null mutation were also observable in PV-Cre;ErbB4−/− mice where ErbB4 is specifically knocked out in PV-positive interneurons (FIG. 14 and FIG. 16). In addition, PV-Cre;ErbB4−/− mice are impaired in working memory (FIG. 15), in support of the ideas that PV-positive interneurons are important in modulating cognitive processes and that disturbance in GABAergic neurotransmission could be a pathologic mechanism of schizophrenia (Markram, et al., Nat Rev Neurosci, 5:793-807 (2004); McBain and Fisahn, Nat Rev Neurosci, 2:11-23 (2001); Lewis, et al., Nat Rev Neurosci, 6:312-324 (2005); Lewis and Gonzalez-Burgos, Nat Med, 12:1016-1022 (2006)). These observations are consistent with the notion that pharmacological alteration of GABA inhibition onto pyramidal neurons may be beneficial for cognition deficits in schizophrenia (Lewis, et al., Am J Psychiatry, 165:1585-1593 (2008)).

In agreement, diazepam attenuates the PPI disruption in PV-Cre;ErbB4−/− mice (FIG. 16C), indicating that altered GABAergic neurotransmission may account for at least in part behavioral deficits in the mutant mice. Together, these results suggest that PV-positive interneurons are a major cellular target of abnormal NRG1/ErbB4 signaling in schizophrenia. They are in line of the idea that disrupted NRG1 signaling may cause imbalance in neuronal activity in the brain, providing insight to possible pathogenic mechanisms of schizophrenia. Finally, unlike NRG1 and ErbB4 null mutant mice that often die prematurely, PV-Cre;ErbB4−/− mice survive into adulthood and thus could serve as a valuable model to study schizophrenia and relevant brain disorders.

We claim:

1. A pharmaceutical composition comprising an ErbB4 antagonist ligand in an amount effective to decrease GABAergic transmission in a subject.

2. The pharmaceutical composition of claim 1 wherein the ErbB4 antagonist ligand comprises soluble ErbB4.

3. The pharmaceutical composition of claim 2 wherein the ErbB4 antagonist ligand promotes a stimulatory response in host by reducing or inhibiting GABAergic transmission.

4. The pharmaceutical composition of claim 1, wherein the ErbB4 antagonist ligand comprises a small molecule ligand.

5. The pharmaceutical composition of claim 4, wherein the small molecule ligand inhibits the interaction between NRG1 and ErbB4.

6. The pharmaceutical composition of claim 1 further comprising a second therapeutic agent.

7. The pharmaceutical composition of claim 6 wherein the second therapeutic agent is selected from the group consisting of diazepam, methamphetamine, amphetamine and dextroamphetamine, gabapentin, potassium chloride, methylphenidate, clonazepam, modafinil, lamotrigine, aripiprazole, triamcinolone, valproate semisodium, divalproex sodium, phenyloin sodium, lithium, natalizumab, promethazine, reperidone, temazepam, topiramate, prednisone, triamcinolone, verapamil, or combinations thereof.

8. A method for treating a neurological disorder comprising administering to subject in need thereof an effective amount of a pharmaceutical composition comprising of an ErbB4 antagonist ligand to inhibit or reduce the disorder.

9. The method of claim 8, wherein the neurological disorder is selected from the group consisting of schizophrenia, epilepsy, depression and anxiety, insomnia, stroke, pain, bipolar, autism, or a combination thereof.

10. The method of claim 9 wherein the ErbB4 antagonist ligand is in an amount effective to reduce or inhibit GABAergic transmission relative to a control, wherein the decrease in GABAergic transmission induces a stimulatory effect in the subject.

11. A method for inducing a stimulatory effect in a subject comprising administering to the subject an effective amount of an ErbB4 antagonist ligand.
